(19) United States
(12) Patent Application Publication
(10) Pub. No.: US 2023/0082022 A1
(43) Pub. Date: Mar. 16, 2023

(54) SYSTEM AND METHOD FOR CONSTRUCTION AND IMPLEMENTATION OF AN ELECTRICAL STIMULATION ENHANCED SURGICAL IMPLANT

(71) Applicant: Intelligent Implants Limited, Cork (IE)

(72) Inventors: Erik Robert Zellmer, Gothenburg (SE); Christian Franz Berkius, Gothenburg (SE)

(21) Appl. No.: 17/942,986

(22) Filed: Sep. 12, 2022

Related U.S. Application Data
(60) Provisional application No. 63/242,985, filed on Sep. 10, 2021.
(60) Provisional application No. 63/244,608, filed on Sep. 15, 2021.
(60) Provisional application No. 63/245,086, filed on Sep. 16, 2021.
(60) Provisional application No. 63/305,999, filed on Feb. 2, 2022.

(51) Int. Cl. A61N 1/05; H01R 13/10; H01R 13/05; H01R 24/00

(52) U.S. Cl. CPC A61N 1/05; H01R 13/10; H01R 13/052; H01R 24/00; A61N 1/0551; A61B 17/72

(57) ABSTRACT

A system and method for an electrically enhanced surgical implant comprising: an implant body that includes an inner frame, wherein the inner frame includes a set electrode sites, and an over-coating that is formed over the inner frame, leaving the electrode sites exposed on the surface of the implant body; a circuitry casing, electrically and mechanically connected to the implant body, implant circuitry, situated at least partially within the implant casing, comprising receiver circuitry, effective to convert an electromagnetic field to electric current, control circuitry, and a power source; a set of conductive paths, wherein each conductive path has a first portion, electrode, situated on an electrode site, and a second portion, electrical conduit, that extends on and through the inner frame and electrically connects the electrode to the implant circuitry in the circuitry casing. The system functions as an electrically enabled surgical implant, such that the surgical implant can provide precisely determined and localized electrical stimulus as part of the implant operation.

### CROSS-REFERENCE TO RELATED APPLICATIONS

This application claims the benefit of U.S. Provisional Application No. 63/242,985, filed on 10 Sep. 2021, U.S. Provisional Application No. 63/244,608, filed on 15 Sep. 2021, U.S. Provisional Application No. 63/245,086, filed on 16 Sep. 2021, and U.S. Provisional Application No. 63/305,999, filed on 2 Feb. 2022, all of which are incorporated in their entireties by this reference.

### TECHNICAL FIELD

This invention relates generally to the field of surgical implants, and more specifically to a new and useful system and method for construction and implementation of an electrical stimulation enhanced surgical implant.

### BACKGROUND

Orthopedic surgery is one of the most common branches of surgery performed within the US and in Europe, where orthopedic surgeons use many means to treat musculoskeletal trauma, spine diseases, sport injuries, degenerative diseases, infections, tumors, and congenital disorders. Orthopedic implants have been used in many situations to introduce, replace, or connect tissue. Most of these orthopedic implants have been simple structural constructions that have aided medications, treatments, and the regenerative mechanisms of the body to treat disorders.

Often, these orthopedic implants are surgically placed in ideal/appropriate positions in a patient to be able to provide further treatment, without the capability to do so. Electrical stimulation in tissue (particularly bone tissue) is known to alter tissue growth (enable tissue growth or decay), in addition to being able to alter other body physiological responses. While there has been some exploration in having orthopedic implants that can controllably provide electrical stimulation treatment, designing and manufacturing such a medical device has many challenges. Such devices have to incorporate electronic components, with significant limitations on space, while also serving as a structural element that is resistant to the forces encountered as an orthopedic implant. Thus, there is a need for a general implementation of implants that are capable of providing controlled electrical stimulation. This invention provides such a new and useful system and method.

### BRIEF DESCRIPTION OF THE FIGURES

FIGS. 1 and 2 are a schematic representation of an example spinal cage surgical implant system.

FIG. 3 is a schematic of an example surgical nail implant system.

FIG. 4 is a schematic representation of an example solid surgical nail implant system segment.

FIGS. 5-7 are pictures of an example construction of connector piece holes.

FIG. 8 is an example schematic for a folded PCB containing implant circuitry.

FIGS. 9-11 is an example illustration for packing a flexible PCB into a circuitry casing.

FIGS. 12-13 is an example illustration for packing a semi-flexible PCB into a circuitry casing.

FIGS. 14-15 is an example illustration for packing rigid PCBs into a circuitry casing.

FIGS. 16 and 17 are pictures of a sample prototype implant body with electrical connection between antennas and connector piece holes.

FIGS. 18-21 are schematics of an example overmolding process of electrical conduits.

FIG. 22 is a schematic example construction of electrical conduits and electrode sites.

FIGS. 23-25 are examples of other manufacturing and design variations.

FIGS. 26-28 are sample illustrations of different spinal cage surgical implants.

FIGS. 29-38 are example illustrations of spinal cage implant body components.

FIG. 39 is an example prototype of a comparison between a fully connected inner frame with a connector piece and a fully connected inner frame with a connector piece overmolded with an over-coating.

FIG. 40 is an example illustrations of a connected inner frame with a connector piece.

FIGS. 41-42 are images of an example prototype of a connected inner frame with a connector piece.

FIG. 43 is an example illustration of an overmolded implant body with a connected casing.

FIG. 44 is an example of a circuitry casing at different levels of assembly.

FIGS. 45-46 are example illustrations of a spinal cage with a connected circuitry casing.

FIGS. 47-50 are example illustrations of circuitry casing components.

FIG. 51 is a sample prototype of the implant body and circuitry casing for a spinal cage.

FIGS. 52-55 are illustrations of an example construction of an electrical conduit between antennas and connector holes.

FIG. 56 is a picture of an example surgical nail implant.

FIG. 57 is a schematic of an example head region of a surgical nail.

FIG. 58 is a schematic example of an circuitry casing connection to the surgical nail head region.

FIG. 59 is a schematic of a surgical nail with a solid inner frame.

FIG. 60 is a schematic of a surgical nail with a tubular inner frame.

FIG. 61 is a schematic of a surgical nail with an open inner frame.

FIG. 62 is a schematic of a surgical nail that has an inner frame that is partially tubular, partially solid, and partially open.

FIG. 63 is a schematic of an open segment of a surgical nail with electrode stimulation sites on the exterior of the surgical nail.

FIG. 64 is a schematic of an open segment of a surgical nail with an electrode stimulation site proximal to the opening of the surgical nail.

FIG. 65 is a schematic of an open segment of a surgical nail with an electrode stimulation site along the interior of the surgical nail.

FIG. 66 is a flowchart of an example method.

FIGS. 67-68 show one implementation of electrodes incorporated by sputtering.

### DESCRIPTION OF THE EMBODIMENTS

The following description of the embodiments of the invention is not intended to limit the invention to these embodiments but rather to enable a person skilled in the art to make and use this invention.

### 1. Overview

A system and method for construction and implementation of an electrically stimulating surgical implant includes: preparing an implant body; wherein the implant body comprises a surgical implant composed of conductive and/or non-conductive materials; preparing the implant body for electrical components, which can include adding a base insulation layer to separate the electrical components from the implant body conductive material, and establishing electrode sites and establishing electrode conduits on and within the implant body; installing the electrical components, which includes attaching electrodes and their electrical connections on and within the implant body; finishing the implant body, which includes adding an outer insulation layer to at least partially cover the implant body electrical components; building an implant casing, that includes implant control, power, and communication circuitry; and connecting the casing to the implant body.

The system and method function to create an “enhanced” surgical implant with electrodes and electronics integrated through the manufacturing design of the device. The integrated electrodes may be used in providing electrical stimulation capabilities and/or electrode-based sensing, and/or other electronics-based capabilities. The system and method function to construct and implement an electrically stimulating implant or to add electrically stimulating functionality to the implant. In this manner, the system and method may be implemented as their own unique process and/or construction; or in conjunction with the construction and/or implementation of a distinct implant. Additionally or alternatively, the system and method may enable integration of other electrical components with a surgical implant. In this manner, the system and method function to securely integrate electronics into the structural elements of a biomedical device.

The system and method may be implemented with any general implant and/or any general implant construction method to create an enhanced electrically stimulating implant. The system and method may be applicable for the use and construction of surgical implants made of either conductive or non-conductive materials (e.g., titanium, poly-ether ether ketone, silicone, bio-material, etc.).

The system and method may be particularly applicable to the field of orthopedic implants, wherein the system and method may be implemented for the construction of surgical nails (i.e., intermedullary nails) and spinal cages (e.g., lateral cages).

The system and method may provide a number of potential benefits. The system and method are not limited to always providing such benefits, and are presented only as exemplary representations for how the system and method may be put to use. The list of benefits is not intended to be exhaustive and other benefits may additionally or alternatively exist.

One potential benefit of the system and method is to provide a relatively easy implementation of electrical stimulation with surgical implants. That is, the system and method provide a reliable manufacturing process for the implementation of electrical components within the structural components of surgical implants.

Additionally, the system and method potentially provide the benefit of incorporating toxic electronic components in a safe manner for a biological implant. That is, the system and method enable use and construction of a hermetically sealed casing that effectively isolates potentially hazardous electronic components while still enabling their use with a biological implant.

The system and method may provide an “enhanced” implant that may potentially provide a better treatment as compared to a similar non-stimulating implant. By providing electrical treatment, an orthopedic implant potentially provides an improved treatment. This improved treatment may be implementation dependent. Examples of improved treatment may include: modifying tissue growth (e.g., increasing/decreasing bone growth) and adjusting biological behavior (increasing/decreasing heart rate, increasing/decreasing blood flow, increasing/decreasing gastrointestinal motility).

Additionally, the system and method may provide an improved method for tissue monitoring. Through use of the system and method, bone growth, and other tissue growth, may be more precisely monitored without the incorporation of the use of additional invasive procedures beyond inserting the implant. For example, impedance measuring capabilities enabled through dynamic control of the electrodes may enable monitoring of bone growth and/or tissue density in surrounding proximity of a surgical nail.

The system and method may also enable incorporation of other sensors within and along the orthopedic implant (e.g., temperature and stress sensors). These sensors may further improve the effectiveness of the surgical treatment by enabling more precise observation and response within the implant that does not require invasive measurement techniques.

### 2. System

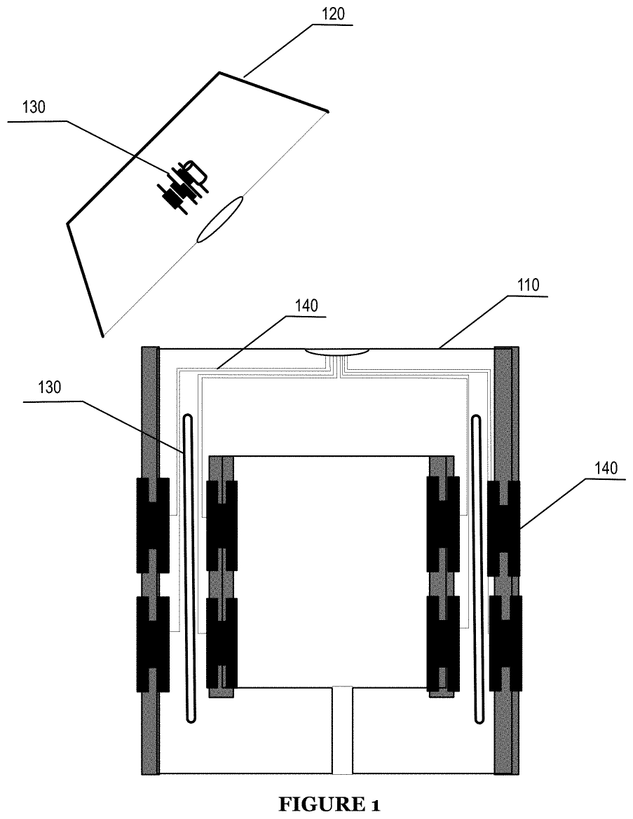

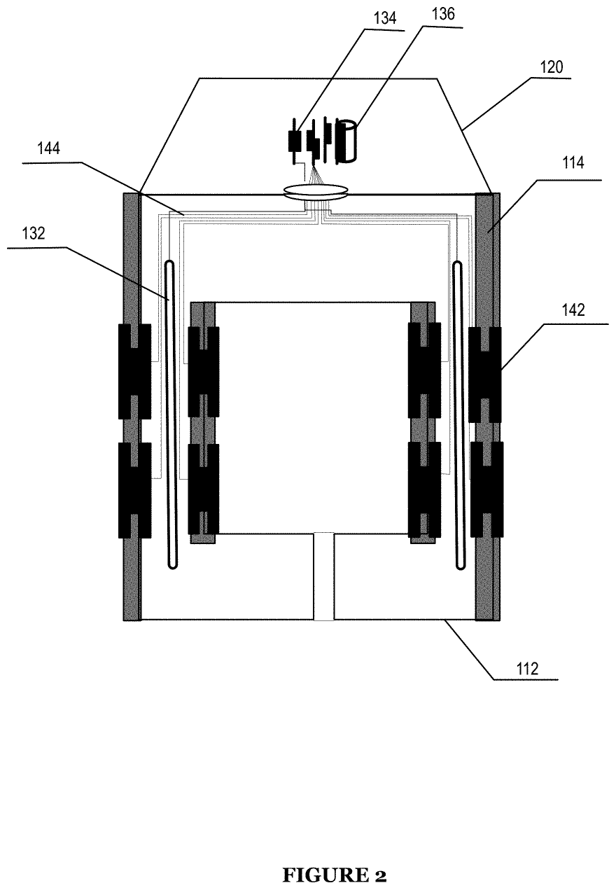

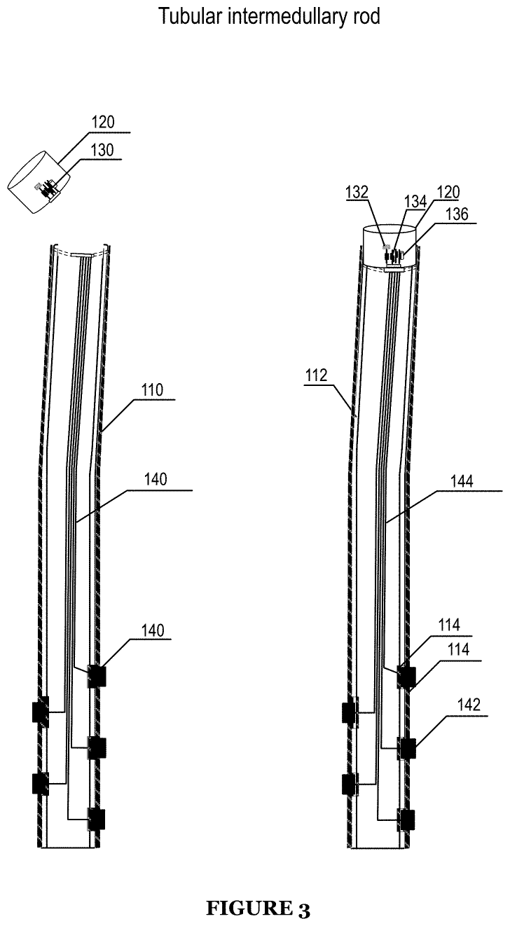

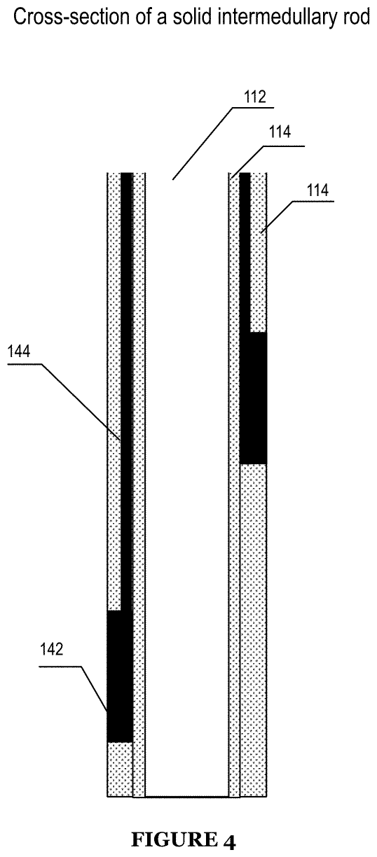

As shown in FIGS. 1 and 2, a system for an electrically enhanced surgical implant includes: an implant body 110 comprising an inner frame 112, wherein the inner frame includes a set electrode sites and an over-coating 114, that is formed over the inner frame, leaving the electrode sites exposed on the surface of the implant body; a circuitry casing 120, electrically and mechanically connected to the implant body, implant circuitry 130, situated at least partially within the implant casing, comprising: receiver circuitry 132, effective to convert an electromagnetic field to electric current, control circuitry 134, and a power source 136; a set of conductive paths 140, wherein each conductive path has: a first portion, electrode 142, situated on an electrode site, and a second portion, electrical conduit 144, that extends on and through the inner frame 112 and electrically connects the electrode to the implant circuitry in the circuitry casing. The system functions as an electrically enabled surgical implant, such that the surgical implant can provide precisely determined and localized electrical stimulus as part of the implant operation. Dependent on implementation, this electrical stimulus may be the primary or a secondary function for the implant. For example, in one implementation the system may comprise a pacemaker surgical implant, wherein the primary function of the implant is to provide regular electrical stimulus to stimulate the heartbeat of a patient. In another example, as shown in FIGS. 3 and 4, the system may comprise an intermedullary rod, with a primary function to hold tissue together (e.g., bone tissue). Additionally, the electrically enhanced intermedullary rod may provide electrical stimulation (e.g., to stimulate and/or monitor tissue growth around the implant).

As an electrically enabled device, the surgical implant may have at least two primary embodiments: a first embodiment, wherein the surgical implant is primarily composed of non-conductive material; and a second embodiment, wherein the surgical implant is primarily composed of conductive material. As shown in FIGS. 1 and 2, embodiments where the inner frame 112 is composed of primarily non-conductive material (e.g., plastics such as PEEK), the system may require only a single over-coating “layer” (although multiple layers may be implemented as desired). As shown in FIGS. 3 and 4, in embodiments where the inner frame 112 is primarily composed of conductive material (e.g., titanium, platinum, etc.), the system may include multiple over-coating “layers” wherein these layers separate the set of conductive paths 140 both from the conductive inner frame 112 and from external components. Dependent on implementation, a combination of the teachings of these two embodiments may enable implementation of the system as part of any surgical implant, regardless of the implant composition. That is, the electrically enabled surgical implant may be implemented in conjunction with any general surgical implant, therein providing enhanced functionality to that implant.

In one variation, a system for a bio-implantable stimulation device can include: a circuitry casing 120 comprising control circuitry 134 and a casing connector (also called connector piece); an inner frame 112 comprising a set of electrode sites; a set of conductive paths 140 on the inner frame, where each conductive path has a first portion (i.e., electrode 142) on a surface of the electrode site and a second portion formed as an electrical conduit 144 connecting the electrode site to a connector of the casing connector; and an over-coating 114 that is formed around the inner frame 112 with the electrode sites exposed on a surface of the over-coating. This system may be used to form a medical implant device that can have one or more electrode sites integrated into the construction of the device. Additionally, the system can be designed such that less biocompatible elements (e.g., circuit elements like integrated circuits or active circuit elements like resistors, transistors, capacitors etc.) are securely shielded and contained within a circuitry casing 120. Conductive circuit elements (e.g., electrode sites, wire coils, antennas, etc.) that functionally benefit from being exposed outside of the circuitry casing 120 are integrated into the structural form of the device.

In some variations, the circuitry casing 120 engages with the main implant body 110 (e.g., the inner frame 112) via a connector. This design can make a more manufacturable and/or otherwise enhanced design. In one such variation, a system for a bio-implantable stimulation device may more specifically include: a circuitry casing 120 with a casing connector; an inner frame 112 comprising a set of electrode sites, where the set of electrode sites are raised platform structures; a body connector attached to one end of the inner frame and that electrically couples to the casing connector; a set of conductive paths on the inner frame, where each conductive path has a first portion on a surface of the electrode site and an electrical conduit 144 portion connecting the electrode site to a connector of the body connector and thereby connected to the control circuitry 134 through the casing connector; and over-coating 114 that is formed around the inner frame 112 with the set of electrode sites exposed on a surface of the over-coating 114.

Accordingly, in some variations, the system can include a body connector. The body connector may be attached to one end of inner frame 112. In this variation, the second portion of each conductive path (e.g., the electrical conduit 144 portion) is formed as an electrical conduit 144 on the inner frame 112 from the electrode site to a conductive contact point of the body connector; wherein the casing connector mechanically and conductively couples to the body connector. The body connector and the casing connector can include multiple contact points, which may be formed as pins, complimentary male and female electrical connectors, or any suitable electrical contact elements. In some variations, the circuitry casing 120 can be welded, fused, or otherwise attached to the body connector. The mechanical connection may be a non-reversible connection such that the circuitry casing 120 becomes substantially permanently integrated with the rest of the implant body 110. For example, the circuitry casing 120 can include a metal casing containing the control circuitry 134 and any other circuitry elements. The body connector can include an outer metal ring or surface. The metal casing can be welded to the metal surface of the body connector. This can form a hermetic seal and also form a substantially permanent connection between the body connector and the casing connector. The body connector can be integrated with the inner frame 112 the implant body 110 more broadly in a variety of ways. In one variation, the body connector can physically couple with the inner frame 112. For example two side panels of the inner frame 112 may clamp around a portion of the body connector. Then, in some variations, the over-coating 114 when formed around the inner frame 112 can encase and substantially permanently integrate the body connector with the inner frame 112 and the rest of the implant body 110.

The form of the inner frame 112 can include physical design features that enable integration of electronics elements into the body. To enable a set of electrodes 142 that have stimulation points exposed at one or more locations of the device, the set of electrode 142 sites can be raised platform structures on the inner frame 112. The raised platform surfaces can have one “top” surface or raised surface that has a form in the shape of the targeted electrode 142. For example, the raised platform can be a rectangle or a circle, but may alternatively be ay suitable shape. The raised platform can have slopped path from the top surface to a lower surface, which functions as continuous surface so that conductive path can flow from an eventually exposed electrode site down to an eventually covered layer of the device. In one such variation, the raised platform structures can have a ramp from a top surface of the raised platform structure to a recessed surface of the inner frame 112. Additionally, the second portion of the conductive path can run from the recessed surface up the ramp to the top surface of the raised platform structure. The second portion here can be formed as an electrical conduit 144 connecting the electrode site to a connector like the body connector. When the circuitry casing 120 is attached, the body connector conductively couples to a casing connector of the circuitry casing 120 and then to the control circuitry 134.

As discussed, in some variations, the circuitry casing 120 can include a metal body, and the control circuitry 134 can be contained within the outer metal body. The control circuitry 134 can be arranged as a folded circuit system, where different subsections of the circuitry can be arranged in layers. This folded circuit system configuration can function to enable the circuitry to be arranged in a compact volume. In some variations, the control circuitry 134 can be a partially flexible PCB (printed circuit board), a flexible PCB, or an integrated stacked PCB configuration.

In some variations, the over-coating 114 can be a material molded around at least a portion of the inner frame 112. The inner frame 112 can form the skeleton with the overcoating forming a structural outer later in some variations. The over-coating 114 is layered around the inner frame 112 with the electrode sites at least partially exposed. Because the electrode sites may be formed as a raised surface, the electrode sites may be positioned substantially coplanar with an outer surface of the over-coating 114. In one variation, the over-coating 114 is injected molded using a material compatible with injection molding. The over-coating 114 in one variation can be polyetheretherketone (PEEK). Other materials may alternatively be used. In another variation, the over-coating 114 could be an insulating material or layer that is deposited or otherwise formed around the inner frame 112. This may be used in devices where the inner frame 112 is substantially responsible for the structural integrity, like for a surgical nail where the inner frame may be a metal structure.

In some variations, the device can include one or more metal coils which can be used as antennas, transmitters or receivers, inductors or the like. In such a variation, the inner frame 112 can include at least one antenna coil inset; and a wire coiled around the antenna coil inset, wherein two ends of the wire are conductively connected to a connector (e.g., the body connector or the casing connector) and thereby to the control circuitry 134. The wire will generally be a metal wire where a first end may be connected to a first connector port of a body connector and a second end is connected to a second connector port of a body connector. Then the two ends of the wire are conductively connected when the casing connector mechanically and/or conductively couples to the body connector. In this variation, connecting a body connector to a casing connector can establish conductive connections (e.g., paths) between the control circuitry 134 and one or more antennas and/or one or more electrode sites.

In some variations, defined tunnels can be formed with the inner frame 112 to enable paths to navigate from one surface of the inner frame 112 to a connector. Accordingly, for one electrode 142, the inner frame 112 can include a defined channel tunnel from a surface of a first electrode site of the set of electrode sites to a connection point to the casing connector, wherein a first conductive path of the set of conductive paths is conductively connected from the first electrode site to the connection point through the defined channel tunnel. Multiple electrode sites may use defined channel tunnels to navigate a path a from an electrode site to a conductive connection to the control circuitry 134.

In some variations, defined tunnels may be used to facilitate connecting or bridging one end of an electrical conduit 144 from one surface to a connector. Accordingly, in one variation, a body connector can be attached to one end of the inner frame 112; and the second portion of each conductive path can be formed as an electrical conduit 144 on the inner frame from the electrode site to a conductive contact point of the body connector; wherein the casing connector mechanically and conductively couples to the body connector; and the defined channel tunnel can extend from one end of the second portion of the first conductive path to a first conductive contact point of the body connector. The tunnel can function as an intermediary path from the end or a subsection of the second portion to a connection point that connects to the casing connector (and thereby the control circuitry 134). In other words, a conductive path can flow from an electrode site, down along an electrical conduit 144 portion, and then through the defined tunnel to a connector.

In some variations, defined tunnels may be used to bridge an electrode site on a first surface to an electrical conduit 144 on a second defined surface. In such a variation, the system may include a body connector attached to one end of the inner frame 112; and wherein the second portion of each conductive path is formed as an electrical conduit 144 on the inner frame 112 from the electrode site to a conductive contact point of the body connector; wherein the casing connector mechanically and conductively couples to the body connector; and wherein the defined channel tunnel is defined from a first side of the inner frame 112 where the first electrode site is positioned to a second side of the inner frame where the second portion of the first conductive path is positioned, wherein a conductive connection is established between the first electrode site to the second portion of the first conductive path through the defined channel tunnel. The tunnel can function as an intermediary path from an electrode site to another side of the inner frame 112 where the electrical conduit 144 portion is substantially positioned. This may be used to keep the electrical conduits 144 grouped and/or positioned on a limited number of surfaces.

In some variations, the conductive path can be formed through a defined tunnel in ways substantially similar ways to how the conductive paths are formed elsewhere (e.g., patterned sputtering or adhering foil). In some variations, the system can include conductive epoxy deposited into the defined channel tunnel, wherein the conductive connection from the first electrode site to the connection point through the defined channel tunnel can be formed by the conductive material deposited into the tunnel. The material could be conductive epoxy, but other conductive materials may alternatively be deposited or injected into the tunnel.

In some variations, the inner frame 112 can include structural elements to guide or reinforce the conductive paths. In one variation, the inner frame 112 can include a recessed channel defining a path between a first electrode site of the set of electrode sites to a connection point, wherein for a first conductive path of the set of conductive paths, the second portion of the first conductive path is formed by conductive material deposited into the recessed channel.

The conductive paths may be formed in a variety of ways. In one variation, the set of conductive paths can be a conductive layer sputtered onto the inner frame 112. In another variation, the set of conductive paths may be patterned conductive foil adhered to surfaces of the inner frame 112.

In some variations, the inner frame 112 may be made of a conductive material. For example, a surgical nail may include a metal inner frame 112. In this variation, the system may additionally include an insulating layer between the inner frame 112 and the set of conductive paths. In some variations, this insulating layer could be or replace the over-coating 114. The insulating layer may be patterned, adhered, deposited over all of the inner frame 112 or sub-portions, and/or otherwise added to the inner frame 112. In one variation, the insulating layer may be patterned along the paths (typically extending beyond border of conductive paths to ensure electrical isolation.

The system may be decomposed or broken into various sub components. The subcomponents may be combined in any suitable manner to form the structures and systems described herein. In one example, the inner frame 112 may be made of multiple sub components that can be used in combination to form the inner frame 112. Accordingly, in some variations, the inner frame 112 can include at least two side panels that combine to form the inner frame 112.

The system may include an implant body 110. The implant body 110 may have any general function as desired, or required, for a particular implementation. In one variation, the implant body 110 may be any non-biological implant that may be implanted into a living organism (e.g., spinal cage, pacemaker, nail). Examples of possible implant bodies 110 that can be incorporated as part of the system include: orthopedic implants (e.g., spinal cages, surgical nails, rods, plates, discs), stents, dental implants, prosthetics, and pacemakers. The implant body 110 may be generally composed and/or constructed in a similar fashion to non-system versions of the implant. For example: an intermedullary nail may be constructed of primarily titanium, a spinal cage may be constructed of non-toxic plastic (e.g., polyether ether ketone (PEEK)), and an artificial pacemaker may be constructed of titanium. The shape and form of the implant body 110 may be similar, or analogous, to those of passive medical device surgical implants; such as orthopedic implant devices like cervical plates, spinal cages, meshes, and pins. The implant body 110 may include an inner frame 112 and an over-coating 114.

The implant body 110 may include an inner frame 112. The inner frame 112 provides the general structure of the implant body 110. Dependent on implementation, the inner frame 112 may comprise a single structure, or multiple structures, that when combined, form the implant body 110 structure. Dependent on implementation, the inner structure 112 may be composed of a non-conductive material (i.e., first embodiment), a conductive material (i.e., second embodiment), or some combination of conductive materials. As used herein, reference to the inner frame 112 may refer to each individual inner frame component or may refer to the “combined” inner frame structure, that generally defines the shape and basic structure of the implant body 110.

In some variations the inner frame 112 may further include a connector piece. The connector piece may function to enable a physical and/or electrical connection (i.e., wired connection) between the implant body 110 and components within the circuitry casing 120. That is, the connector piece enables an electrical connection between the set of conductive paths 140 and the implant circuitry 130 located within the circuitry casing 120, while maintaining an appropriately desired seal for the circuitry casing. In this manner, the connector piece may include structural components to connect both to the rest of the implant body 110 and to the circuitry casing 120. Dependent on the material type of the circuitry casing 120, the connector piece may be welded or overmolded to the circuitry casing 120. In one example for a spinal cage constructed of PEEK and a titanium circuitry casing 120, the connector piece may serve as an intermediary piece that locks to the spinal cage and is then welded to the titanium circuitry casing 120.

For the spinal cage variation, the connector piece may be made of titanium and may be secured (e.g., anchored or locked into place) to the PEEK implant body 110 during placement of the over-coating 114 (e.g., by overmolding onto sections of the connector piece). In some variations, the connector piece may then serve as an anchor for attaching the circuitry casing 120. In variations where both the connector piece and the circuitry casing 120 are constructed of metal (for example titanium), the circuitry casing 120 may be attached to the connector piece (and thereby securely fastened to the implant body 110) using metal to metal welding (e.g., laser welding). In variations where the circuitry casing 120 and connector piece are attached, at least in part using laser welding, the circuitry casing 120 may be securely attached to the implant body 110 without substantially heating (or damaging) components within the circuitry casing due to the localized heat input of the laser welding process. Furthermore, for such variations, the volume in between the connector piece and the circuitry casing 120 may be sealed (for example hermetically) which may prevent fluid ingress into/onto wires/connectors between the circuitry casing 120 and implant body 110 electrical circuits potentially without the requirements of additional seals.

For surgical nail variations, the connector piece may comprise a screw like piece that fits within the head of the surgical nail, thereby enabling a seal with the implant body 110. A PEEK circuitry casing 120 may then be molded over the connector piece. Alternatively, the connector piece may have a locking mechanism that latches onto the interior or the exterior of the surgical nail.

Additionally, the inner frame 112 may include electrode sites. Electrode sites comprise regions of the inner frame 112 that enable placement of a electrodes 142. Electrode sites may be shaped specifically such that the stimulating region of each electrode 142 is situated securely on the electrode site and exposed to the exterior of the surgical implant, such that the electrode may provide electrical stimulation to regions outside of the implant itself. The size, shape, and location of the electrode sites may vary dependent on implementation. In some variations, the electrode site may comprise regions of the inner frame 112 that protrudes out (e.g., a raised platform) from the rest of inner frame.

The implant body 110 may include an over-coating 114. Dependent on implementation, the over-coating 114 may have different functionalities including: connecting parts of the implant body 110, providing insulation, and providing an external shape to the implant body 110. The over-coating 114 may generally be composed of any set of non-toxic non-conductive materials. In many variations, the over-coating 114 is composed of a non-toxic plastic, such as PEEK. The over-coating 114 may be applied over and through the inner frame 112 (e.g., by overmolding). Dependent on implementation, the over-coating may be used to cover/protect certain regions of the inner frame 112, help connect sections of the inner frame, give the implant body 110 a desired shape (e.g., ridges to increase friction of the implant such that it stays in place), and/or provide an insulation coating for conductive components. In some variations, the system may have multiple layers of over-coating for a single, or multiple purposes. For example, as shown in FIG. 4, in some variations an over-coating 114 layer may be applied over implant sections (e.g., inner frame 112) that are composed of a conducting material, to then enable electrical components to be placed above them, which then may be covered by an additional over-coating layer. It should be noted that although electrode sites on the inner frame 112 may include an over-coating 114 directly on the electrode site, the actual electrodes 142 (situated either on inner frame or the over-coating) will be exposed and not have an over-coating, such that electrical stimulation may be travel out from the implant.

The over-coating 114 (or over-coating layer) may be particularly useful for use as insulation. The over-coating 114, or over-coating layer, functions to electrically separate (i.e., insulate) electrically active/conducting components (e.g., each individual electrode 142, each electrical conduit 144, conducting components of the implant, and any other exposed electric components). Additionally or alternatively, the over-coating 114 layer may function to separate electrical components on the inner frame 112 (e.g., antennas, electrodes 142, electrical conduits 144, etc.) from biological fluid and tissue of the patient. As electrodes 142 may be activated independently, insulation may be particularly significant in preventing or directing current flows in proximity of the surgical implant. For a conductive surgical implant (e.g., composed of titanium), the over-coating 114 may electrically separate the set of conductive paths 140 from the inner frame 112. This over-coating 114 may comprise a coating (e.g., PEEK), on the inner frame 112 (or along each conductive path 140). The insulating over-coating 114 may be composed of any non-toxic, non-conductive material. In some variations, multiple forms of insulation are used. Examples include surface coatings (e.g., polyimide) and wire sheathes. In some variations, coatings are made from material that may stretch, or is somewhat malleable. For example, the surgical nail surface coating may be bent or otherwise reshaped.

The over-coating 114 may be applied to the system during or after assembly of the entire system. For electrodes 142, an over-coating layer 114 may be pre-acquired for (e.g., an over-coating layer may be layered over the inner frame 112), or may shaped around the electrode during electrode deposition into/onto the implant body 110. For example, to prevent electric conduction between the electrode(s) 142 and the implant body 110, a polyimide base over-coating 114 may be applied onto the surgical nail prior to positioning of the electrodes in place. In another variations, over-coating may be fill (e.g., overmolded) around the set of conductive paths 140, covering the electrical conduits 144 and leaving only the electrodes 142. For a primarily conductive surgical implant, the over-coating 114 may comprise an outer insulation layer.

The system may include a circuitry casing 120. The circuitry casing 120 may be electrically connected and mechanically fixed to the implant body 110 and function to provide a housing the implant circuitry 130. Additionally or alternatively, the casing may provide a housing for other components. The positioning of the casing may be implementation specific, and particularly dependent on the shape, type, and desired functionality of the surgical implant. Examples of the locations the circuitry casing 120 may connect to the implant body 110 may be noted in FIG. 1, for a spinal cage, and FIG. 3 for an intermedullary rod.

The circuitry casing 120 may be integrated with rest of the implant body 110 during the manufacturing process. In some variations, the circuitry casing 120 is integrated with the implant body 110 irreversibly, such that the circuitry casing and the implant body effectively become a single structure. Alternatively, the circuitry casing 120 may be incorporated as a distinct body structure that can be connected or disconnected from the implant body 110.

Generally, the circuitry casing 120 composition may be dependent on the implementation. For example, for spinal cage variations, the circuitry casing 120 may be constructed of titanium. Alternatively, for surgical nail variations, the circuitry casing 120 may be constructed of PEEK. Generally, the circuitry casing 120 may be constructed of PEEK, but may alternatively be constructed of alternative materials such as titanium that may or may not be platinized. In some variations, the circuitry casing 120 may be built or molded around the implant circuitry 130.

In some variations, the circuitry casing 120 may not add substantially to the size and/or shape of the surgical implant. That is, the circuitry casing 120 preferably adds no limitations to the desired volume and/or desired shape of the surgical implant. The circuitry casing 120 may be physically adjacent and connected to the implant body 110. The circuitry casing 120, in some variations, may constitute some of the outermost surfaces of the surgical implant. Alternatively, the circuitry casing 120 may be adjoined to the implant body 110 from an interior cavity (e.g., the circuitry casing 120 may be positioned within the internal cage of a spinal cage). Generally, the circuitry casing 120 may be positioned anywhere along the implant body 110 as desired, limited by functionality and size limitations.

In some variations of the circuitry casing 120 may comprise a primary housing module and an end piece. In these variations, the primary housing module and the end piece may come together to form a sealed circuitry casing 120, while the end piece may enable an electrical connection with the implant body 110.

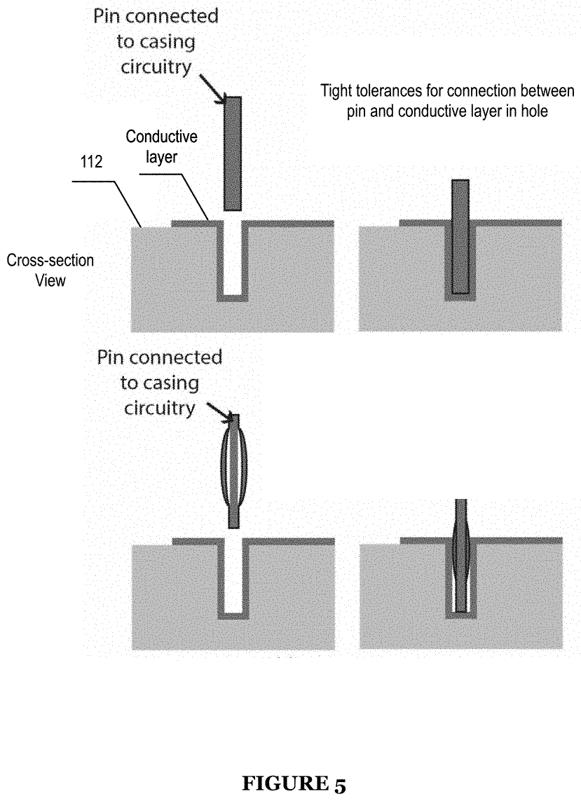

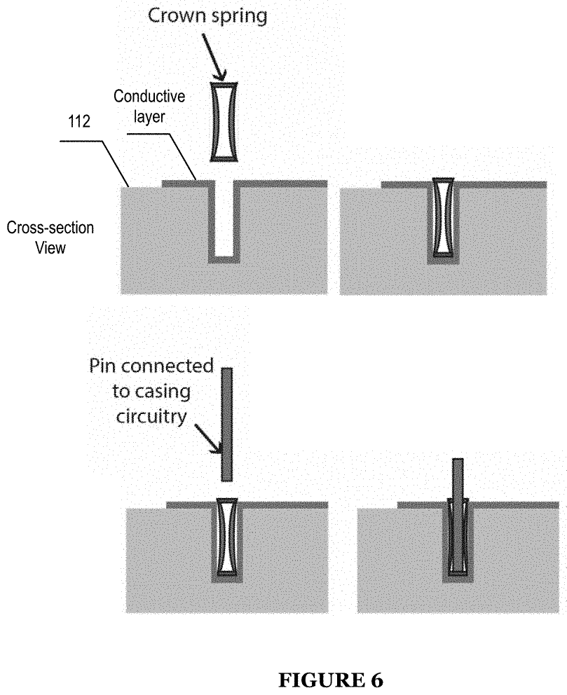

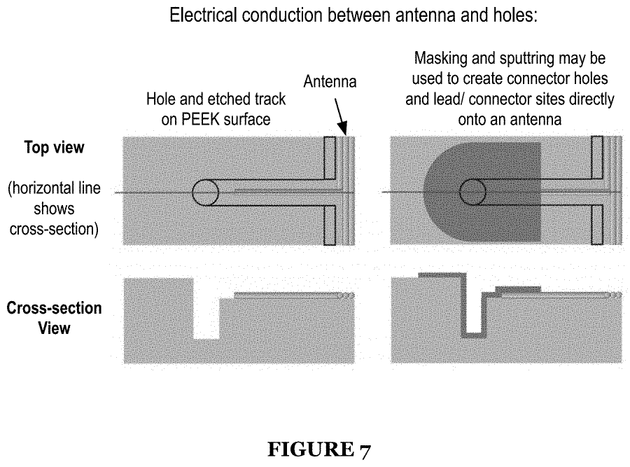

As shown in FIGS. 5-7, masking and sputtering may be used to create connector holes in the inner frame 112. For the circuitry casing 120, as shown in FIG. 5, a pin may be passed through the connector holes to ensure electrical connections between the implant circuitry 130 within the circuitry casing and the implant body 110. Tight or very exact tolerances between a pin and a defined hole may function to establish an electrical connection is formed as shown in the top variation of FIG. 5. In some variations, a pin with a built-in spring (e.g., banana bullet plug connector) may be used to reduce the reliance on very exact tolerances between hole and pin; thus ensuring that electrical connections are formed as shown in the bottom variation of FIG. 5. As shown in FIG. 6, adding a connector piece like a crown spring to a defined connector hole may reduce reliance on exact tolerances between the defined hole and pin as an alternative approach to establishing an electrical connection is formed between a pin and the conductive layer. As shown in FIG. 7, an additional rod-like connector piece (e.g., a crown spring) passed through a connector hole may also reduce the reliance on very exact tolerances between hole and pin, to ensure a reliable electrical connection between the implant circuitry 130 and the set of conductive paths 140. As additionally shown in FIG. 7, an antenna or wire coil could be attached to a connector point. In one variation, attachment of an antenna wire to PEEK may be done using adhesives or other bonding technique. This can be used to attach the antenna to the surface of the inner frame 112. In some variations, laser welding may be used to adhere a wire to a PEEK surface or some other materials surface. This may facilitate connection to a sputtered layer due to, for example, the wire partly melting into the polymer or part of the wire melting both of which may potentially create more favorable connection geometries.

In some variations, the circuitry casing 120 may include a secondary housing module. The secondary housing module may function to provide a distinct housing for specific components that may be disrupted within the first module (e.g., an antenna receiver). In some variations, the circuitry casing 120 may include multiple secondary housing modules. The secondary housing module(s) may be particularly useful in situations where certain components cannot function in the initial casing (e.g., a metallic casing may prevent the function of antenna). This distinct housing may be to separate electrical components such that they do not interfere with each other, and/or to provide a “better” position for positionally dependent electronic components (e.g., an antenna). Dependent on the implementation, the secondary housing module may be directly connected to the primary housing module (i.e., the casing as described previously), or the secondary module may be situated on the implant distinctly to the primary housing module, and connected to the implant body 110 in a similar manner as the primary module (i.e., via a connector piece welded and/or molded into position). For example, the secondary housing module may be situated within a tubular region of a surgical nail, on the tip of surgical nail, on the opposite side of the spinal cage (in relation to the primary housing module), or any other region of the surgical implant as desired.

In some variations, the secondary housing module may have a distinct composition. This distinct composition may help improve functionality of internal components. For example, the first housing module may be composed of non-conductive material (e.g., PEEK), to reduce disruption of communication components (e.g., the antenna), whereas the secondary housing module is composed of material to provide of strong support material (e.g., titanium) to provide better stability and protection for the internal circuitry. In this variation, the implant circuitry 130 may include a PEEK portion housing the antenna and a titanium housing the control circuitry 134. The antenna may be conductively connected to other implant circuitry 130 through sealed connectors. The control system may be conductively connected to exposed connectors for conductive coupling to the electrodes 142.

The system may include implant circuitry 130. The implant circuitry 130 is situated, at least partially, within the circuitry casing 120. The implant circuitry 130 functions as the means of controlling operation of stimulation of the surgical implant. The implant circuitry 130 includes: implant receiver circuitry 132, effective to convert an electromagnetic field to an electric current; implant control circuitry 134, configured to control current flow through the set of conductive paths 140, and a power source 136. Dependent on implementation, the implant circuitry 130 may include other components (e.g., sensors).

The implant circuitry 130 may be primarily located within the circuitry casing 120, such that the implant circuitry is protected from biological fluids and the patient is protected from potentially toxic components from the circuitry. In this manner, the implant circuitry 130 may be sealed (e.g., hermetically sealed) within the casing. In some variations, some parts of the implant circuitry 130 may be contained outside of the circuitry casing 120 (e.g., an antenna).

The implant circuitry 130 may include an implant receiver circuitry 132. The implant receiver circuitry 132 may function to send and receive electromagnetic signals both for communication and to provide electricity for the electrode 142 operation. The implant receiver circuitry 132 may enable external communication. That is, the implant receiver circuitry 132 may function to enable communication with the system (and system components), and external components. Additionally or alternatively, the implant receiver circuitry 132 may enable charging or powering of electronic components on, or within, the surgical implant. That is, the implant receiver circuitry 132 comprises circuitry that enables power signal exchange, which functions to enable wireless delivery of power and/or communication with the implant and implant components, wherein an external device transmits power and/or data to the implant. That is, through the implant receiver circuitry 132, the implant, and/or implant components, may be wirelessly charged and/or powered by an external component. Additionally or alternatively, the implant receiver circuitry 132 may enable transmission of data to external components (e.g., external implant circuitry 130, and/or external computing devices).

The implant receiver circuitry 132 may include one or more transmitter and/or receiver elements. Multiple transmitter and/or receive elements may be used. In one variation, these may be oriented in different directions to facilitate wireless coupling in different directions. In one variation, the implant receiver circuitry 132 includes inductive coil(s) for coupling with a complimentary inductive coil of another device. In another variation, the implant receiver circuitry 132 includes one or more RF (Radio Frequency) antenna(s), ultrasonic transducer(s), and/or other wireless power/data transmission elements. The implant receiver circuitry 132 may include at least one antenna. In one variation, the antenna is at least partially embedded in the circuitry casing 120 and enabled to send and receive communication and electric current. In another variation, the antenna is completely located within the circuitry casing 120. Additionally or alternatively, the implant receiver circuitry 132 may include at least one antenna outside of the circuitry casing 120 (e.g., situated within the inner cavity of a tubular shaft of a surgical nail, or situated encircling the side of a spinal cage).

One or more portions of the implant receiver circuitry 132 may be wireless. Alternative implementations may use wired or direct communication. In some variations, data can be communicated through the wirelessly transmitted power signal, thereby enabling simultaneous (or near simultaneous) power and data transfer. For example, a high frequency data signal could be transmitted on top of a lower frequency power signal. The data signal could be decoded or read during conditioning of the received power signal. Data may include various commands relating to operational state directives, stimulation settings, diagnostics settings, communication settings, and/or other suitable commands. Data from the implant receiver circuitry 132 is preferably implant operating data that may include current settings, diagnostics results, monitoring data, stimulation logs, power status, and/or other information. In some variations, data from the implant receiver circuitry 132 may be held in local memory (e.g., as part of the implant control circuitry 134) until successful transfer to external components.

In some variations external components may transfer power and/or data to the implant receiver circuitry 132 using a first dedicated set of tuned antennas. The implant receiver circuitry 132 may transfer data to external components through a second, distinct set of tuned antennas; that is, the implant receiver circuitry 132 may have a set of “sending” antennas and a set of “receiving” antennas. In another variation, external components may transfer data and/or power using a set of tuned antennas and the implant receiver circuitry 132 may transfer data to external components through the same set of tuned antennas using, for example, load modulation.

In some variations, the power transmission can be modulated according to the power received by the implant receiver circuitry 132. For example, if the power supplied is not enough, external components may be instructed to adjust transmission (e.g., increasing transmission magnitude). In another variation, the data is communicated to external components wherein a doctor or processing unit may determine if any changes should be made to the electrical stimulation.

In one example implementation, the implant receiver circuitry 132 comprises a receiver coil (e.g., a tuned air core planar receiver coil); rectifying circuitry; voltage regulator and an implantable transmitter coil. In this example implementation, the receiver coil and an external transmitter coil may form an inductive link where oscillating electric current within the external transmitter coil induces a potential over the tuned receiver coil through inductive coupling. Alternatively, depending on the transmitter type, the implant receiver circuitry 132 may include receivers suitable for receiving RF irradiation, waves generated by an ultrasonic transducer and/or IR. In some variations, AC current in the receiver coil can be converted into DC current using rectifying circuitry. The voltage of the received signal may also be regulated using a voltage regulator. In one embodiment, capacitor(s) may store energy received through a wireless link and use it to meet the power consumption of the system circuitry. In one embodiment, the rectifying circuit may also function as an envelope detector, and the envelope of AC signals transmitted through the wireless link may be used to control the state of one or more of the system components, either directly or indirectly, via the implant control circuitry 134. In various embodiments, load modulation may be used to send data through the wireless link (e.g., from implant to external components).

The implant circuitry 130 may include implant control circuitry 134. The implant control circuitry 134 functions to activate/deactivate, and control the implant circuitry 130. The implant control circuitry 134 may be at least partially embedded in the circuitry casing 120 and electrically connected to the set of conductive paths 140, and to other “controllable” components (e.g., implant receiver circuitry 132, sensors, battery, etc.). In some variations, the implant control circuitry 134 may be an external component (e.g., a computer) that communicates with the orthopedic implant via the implant receiver circuitry 132.

The implant control circuitry 134 may also enable operating modes for the orthopedic implant. These operating modes may be implementation specific. Examples of different types of operating modes may comprise: different types of electrode 142 activation, both for sensory functionality and for providing tissue stimulation; operation of sensor components; application of externally provided stimulation activity (e.g., doctor prescribed); and/or other types of operation. Dependent on implementation, one or multiple operating modes may be active at one time. In this manner, the implant control circuitry 134 may function to provide real time stimulation and monitoring of tissue, wherein complex patterns of activation and deactivation of electrodes 142 and operating modes may be implemented for relatively precise tissue stimulation.

In some variations, the implant circuitry 130 may have a power source 136. The power source 136 functions to provide power for circuitry operation, particularly electrode 142 operation. Dependent on implementation, the power source 136 may comprise one, or multiple, power sources, where each power source may be of the same, or different type. In some variations, the power source may be an internal power source, i.e., located on, or within the implant, preferably within the circuitry casing 120. Additionally or alternatively, the power source 136 may be an external power source; i.e., located external to the implant, either as a separate implant or outside of the body of the patient.

Internal power sources 136 may be housed within the circuitry casing 120 of the surgical implant, but can alternatively be outside of the circuitry casing (e.g., within the tubular section of the surgical nail or embedded within the inner frame 112 of any surgical implant). Examples of internal power sources 136 may include any type of energy storing devices, such as an internal battery (e.g., rechargeable), or capacitor(s). The internal power source 136 may be electrically coupled to the implant control circuitry 134, the implant receiver system 132, the set of conductive paths 140, and/or any other desired system component. In many variations, the system may include an internal power source(s) 136 for regular operation, and an external power source for charging of the internal power source(s).

External power sources 136 may comprise a separate implant and/or a source external to the patient. The external power source 136 may be directly (e.g., by wiring) or indirectly (e.g., by induction) coupled to the implant and the implant circuitry 130. In variations that include a directly coupled external power source 136, the system may further include wiring connected to the end cap circuitry that extends from the implant to the external power source. This may include a shunt containing the wiring traveling from the implant within the patient to a position on the skin of the patient, such that the wiring may be “plugged-in” to charge the internal power sources 136.

To provide efficient positioning and/or connectivity the implant circuitry 130 may be positioned together as a “circuitry surface”. This circuitry surface may enable efficient function of the implant circuitry 130 components and enable efficient positioning within the circuitry casing. In one variation, the circuitry surface may include a printed circuit board (PCB), wherein implant circuitry 130 components are based on or connected to the PCB. In another variation, the circuitry surface may include an integrated chip (IC), wherein implant circuitry 130 components are built onto, or connected to the IC. For example, the circuitry surface may include an application specific integrated chip (ASIC), wherein an antenna (e.g., from the implant receiver circuitry 132) and capacitor components (e.g., from the power system) are built into the chip. In a third variation, the circuitry surface may include a cavity tube, wherein implant circuitry 130 components are embedded within the tube, or wrapped around the tube (e.g., the antenna).

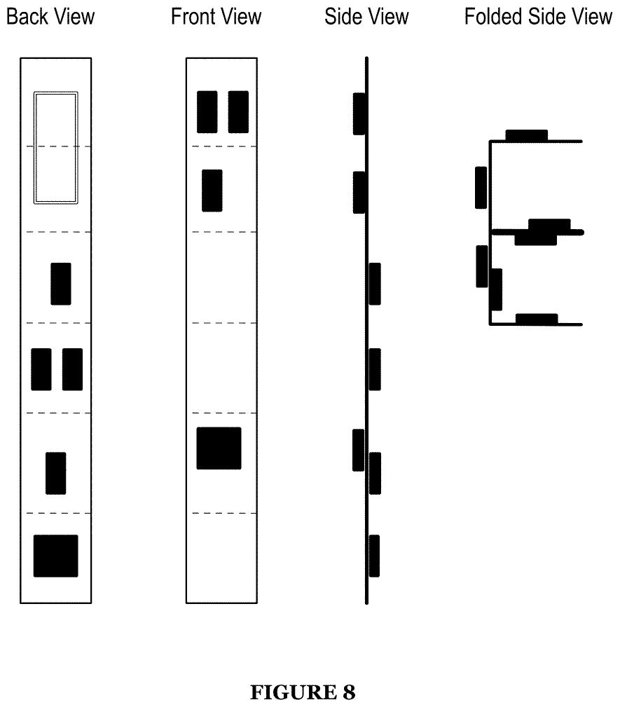

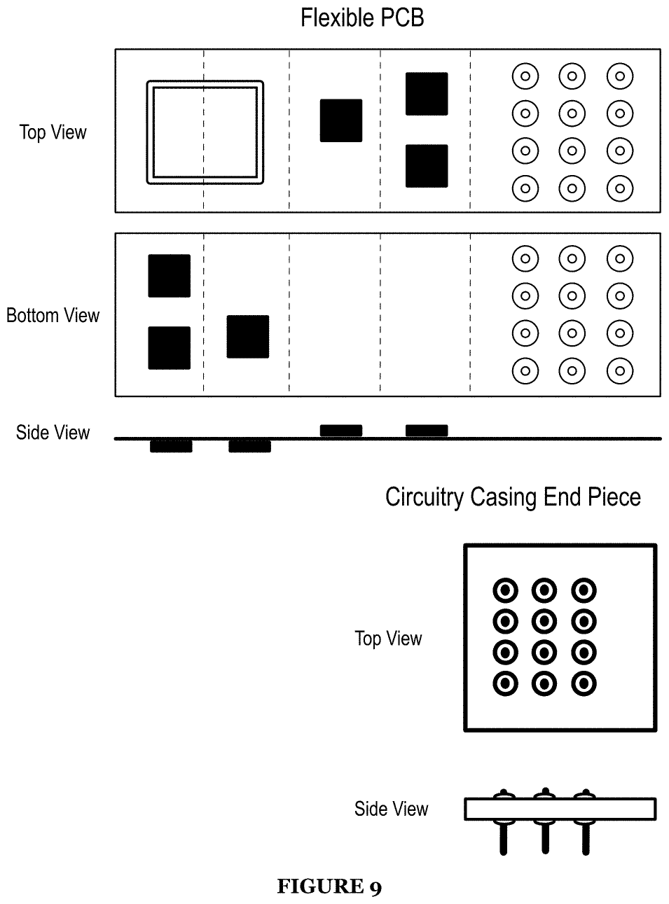

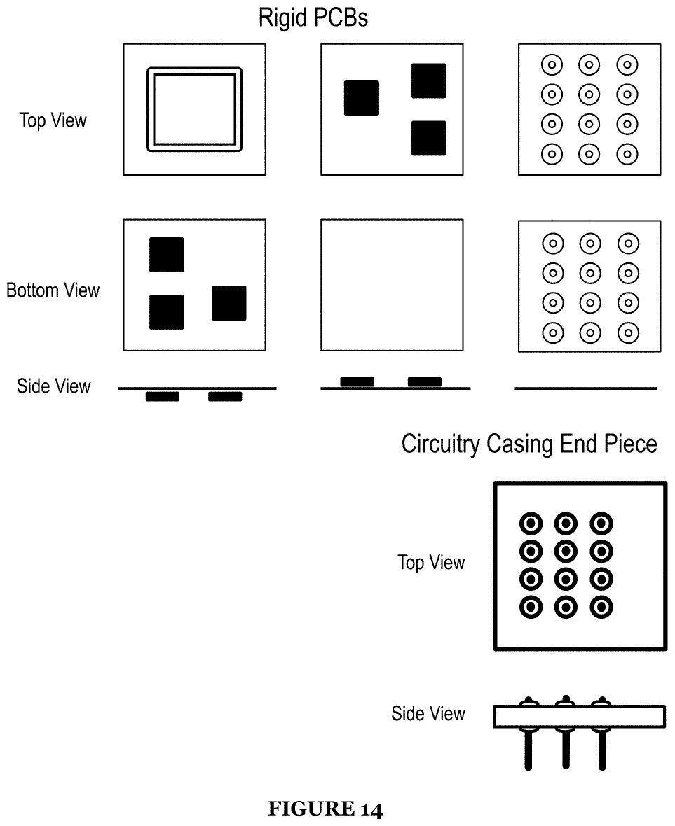

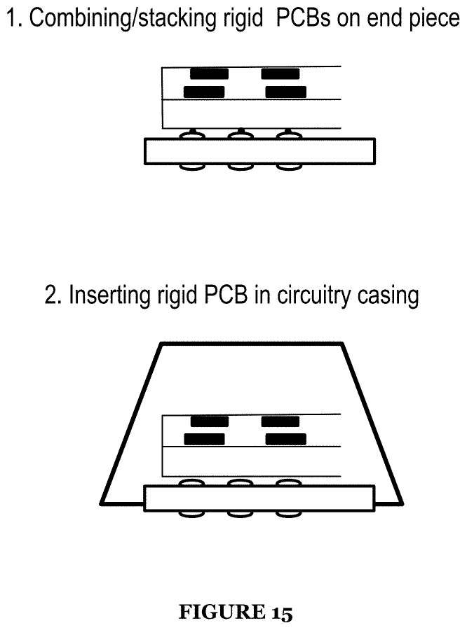

In some variations the circuitry surface comprises at least one PCB as shown in FIG. 8; a schematic drawing of a single layer PCB from a front view (top) and side view (bottom). The PCB may be folded within the circuitry casing 120. PCB folding may function to improve implant circuitry 130 component functionality and to improve the fit of the implant circuitry within the casing. The PCB can be single-sided, double-sided, and/or multi-layered, wherein each side/layer may contain implant circuitry components embedded in, or on, the surface of the PCB. In some implementations, the PCB is single sided and single layered. Dependent on variation, the PCB may be fully flexible, semi-flexible (i.e., have flexible and rigid regions), or be fully rigid, as shown in FIGS. 9, 12, and 14, respectively. In variations, the where the PCB is at least partially flexible (wherein all or parts of the PCB are bendable), the electronic components on the PCB may still not be bendable. In other words, the PCB includes a flexible substrate. Generally, the PCB may include bends and/or folds. The PCB may include any number of bends and/or folds limited such that the final PCB geometry can be incorporated into the casing and that implant circuitry components on the PCB do not lose functionality (e.g., if the component is situated on a PCB bend such that the component is bent beyond function). For implementations that use a rigid PCB, the circuitry surface may comprise multiple smaller PCBs that may be stacked on top of each other.

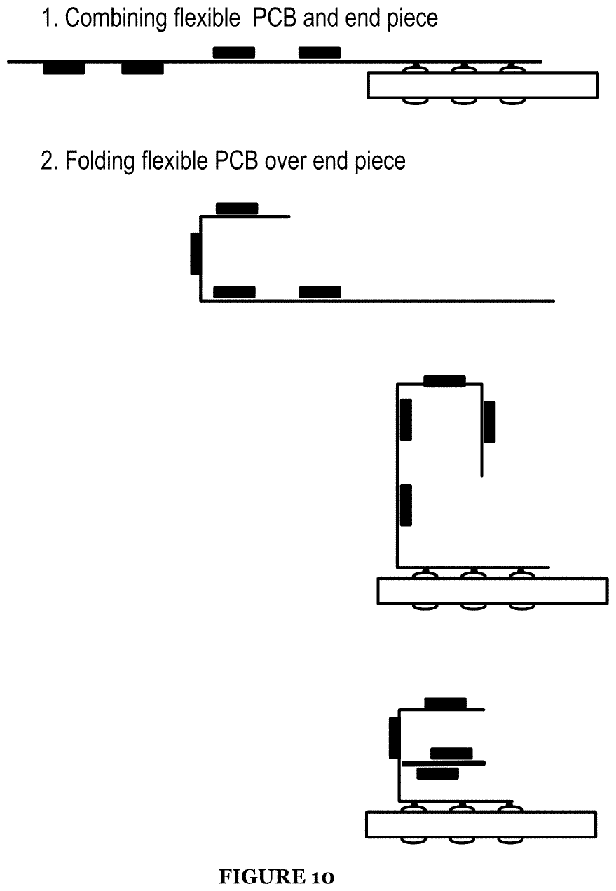

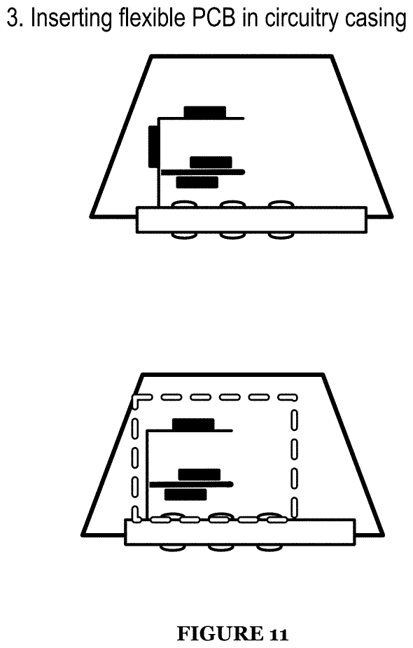

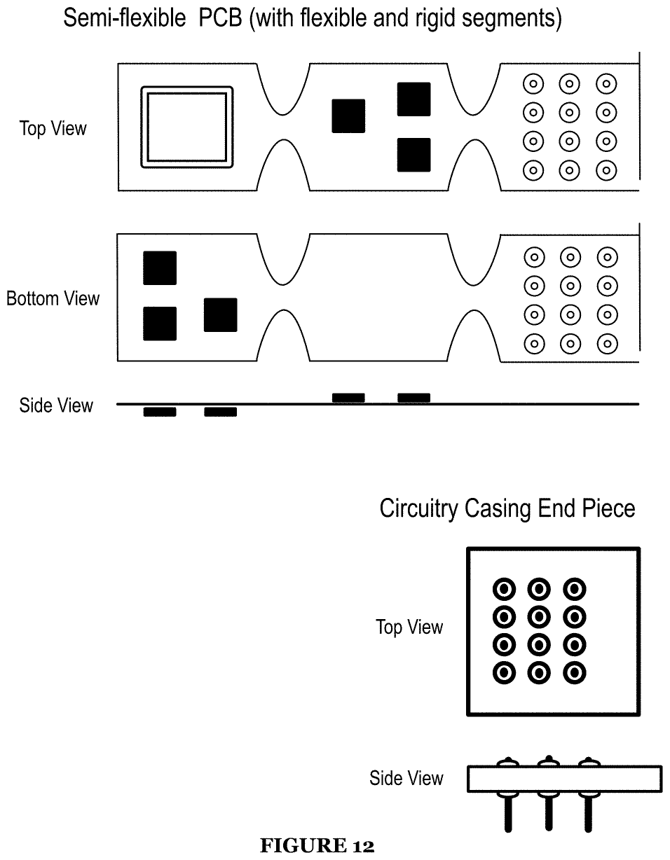

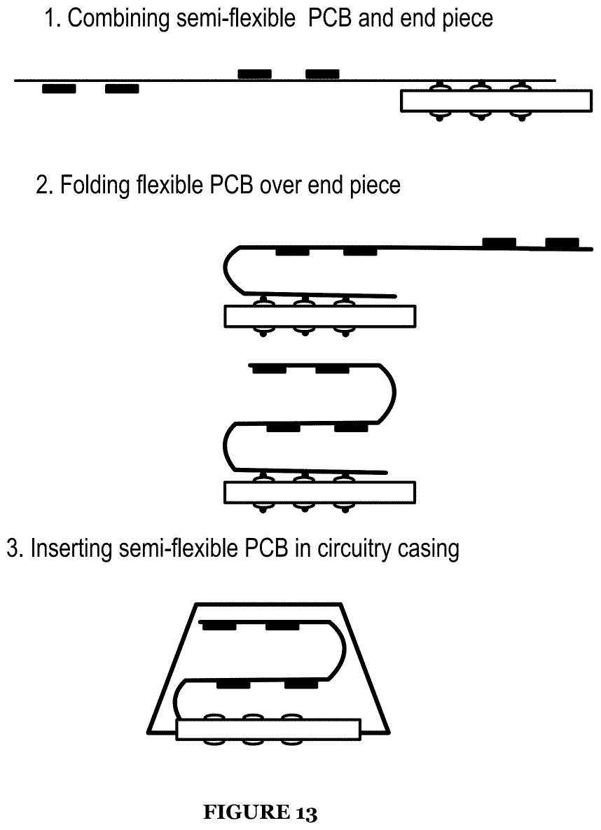

Depending on the type of PCB, flexible, semi-flexible, or rigid, folding of the PCB and fitting it in the circuitry casing 120 may vary. As shown in FIGS. 9-11, for a flexible PCB implementation, the PCB may be initially attached to the circuitry casing end piece. The PCB may then be folded such that entire PCB is over the end piece. Many possibilities may be used in the actually folding configuration for a fully flexible PCB. In one, example, as shown in FIG. 10, the PCB is folded such that the PCB is folded upon itself. In some variations, as shown in FIG. 12 (bottom), prior to placing the PCB into the main body of the circuitry casing 120, the PCB may be initially placed in a secondary casing. This secondary casing may serve to provide greater support for the PCB, and/or may enable separation of the circuitry casing into a secondary housing, which may then be used in the manners described above. As shown in FIGS. 12 and 13, a semi-flexible PCB implementation, the steps for fitting the PCB into the circuitry casing may be similar to the flexible implementation. The semi-flexible may be initially connected to the circuitry casing 120 end piece. The semi-flexible PCB may then be folded over the end piece along the flexible regions of the PCB. Once folded, the PCB is then inserted into the circuitry casing 120. In a third example, for a rigid PCB, multiple PCBs may be used, where each PCB has a smaller cross-section than the end piece, such that they can fit over the end piece. In this example, the rigid PCBs may be stacked on top of each other and combined with the end piece. It may be necessary to place support structures between the PCB stacks, that hold them in place and that enables circuitry to connect each PCB. Once the PCBs are stacked, the end piece may be inserted into the main body of the circuitry casing 120. Example support structures may be pins on the circuitry casing end piece or the PCB, that can be soldered onto receptacles on the PCB after the PCB has been folded to hold together two or more PCB levels or to hold the PCB to the circuitry casing end piece. In one example variation, if the PCB has a half plated or full through-hole on an edge, and the PCB level above or below it has a half-plated hole or full hole on the same horizontal location, when the two levels are placed in the intended configuration in relation to each other, a pin can be used to mechanically attach the two levels. In another variation, if one level of the PCB has a pin mounted on its surface, this may be matched with an hole or half hole on a different PCB level where the positioning of the hole and the pin designates the intended fixation in-between the two levels and where the pin may be soldered in place after passing through the hole to fix the folded structure. In yet another variation, the circuitry casing end piece may have posts that are used during the folding by soldering onto them once a segment has been placed in its correct position.

The system may include a set of conductive paths 140. The set of conductive paths comprise a first portion, electrode 142, situated on an electrode site, and a second portion, electrical conduit 144, that extends on through the inner frame 112 and electrically connects the electrode to the implant circuitry 130 within the implant casing 120.

The set of conductive paths 140 includes electrodes 142. The electrodes 142 function as a first portion of each conductive path wherein the electrode is exposed to the exterior environment of the surgical implant and may provide electrical stimulation (e.g., for treatment). Each electrode is situated on an electrode site on the inner frame 112 (also referred to as a stimulation site). Each electrode 142 may be connected to the implant circuitry 130 via an electrical conduit 144. Each electrode 142 may provide electrical stimulation from the stimulation site to proximal biological tissue. Each electrode 142 may be individually controllable such that any direction current of a desired magnitude may be sent or received from that electrode. Each electrode 142, may also be individually controllable such that the direction of current can be sent or received from each electrode is controlled and the total current magnitude of all sourcing and sinking electrodes also controlled (i.e., current may be distributed over sourcing and sinking electrodes). In this manner, a single electrode 142, multiple electrodes, or the entire set of electrodes may function identically, individually, and/or in a complementary fashion (e.g., one subset of electrodes may be set to function as current sources, sending a current to another subset of electrodes set to function as current sinks.

The shape, size, and number of electrodes 142 (and stimulation sites) may be implementation specific. Variations may depend on the shape, size, type, and purpose, of the surgical implant. In some variations, the stimulation sites may comprise round pads on the exterior of the shaft of the implant body 110. In one surgical nail example of this variation, the surgical nail may have a set of eight round pad electrodes 142, situated on round electrode sites on the inner frame 112, positioned around the shaft. These round electrode 142 pads may be composed of non-toxic conductive material (e.g., titanium). In some variations, the electrode sites on the shaft may be shaped such that the electrode stimulation sites lock into the electrode sites. Alternatively, electrode sites may be molded, adhered, soldered, sputtered, or otherwise attached into each electrode site (potentially with some insulation between the electrode site and conductive regions of the implant body 110). In another variation, the electrodes 142 may comprise flexible/semi-flexible metal plates that are folded through the electrode sites such that they stay fixed in place. In one variation, for an open-section surgical nail region, the electrode 142 may comprise conductive plates directly exposed from the interior of the surgical nail. In another variation, the electrodes 142 may include conductive etchings on the surface of the inner frame 112 (e.g., a conductive etch on the surface of the solid surgical nail.

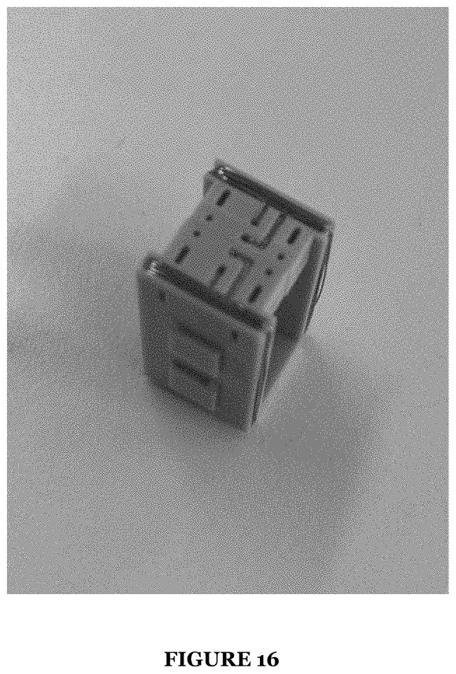

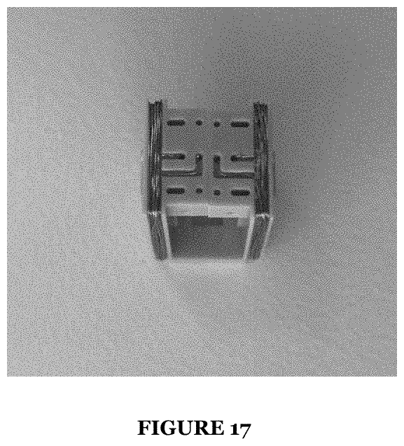

The set of conductive paths 140 includes electrical conduits 144. Electrical conduits 144 electrically connect each electrode 142 to the implant circuitry 130. Additionally, electrical conduits 144 may connect other components to the implant circuitry 130 (e.g., an antenna located on, or within, the implant body 110). As shown in example schematics of FIG. 2 (spinal cage) and FIG. 4 (surgical nail), and the prototype FIGS. 16, 17 (for a spinal cage), etched tracks may create electrical conduits between holes in the implant body 110 or connector piece to the antenna and to electrodes 142. Dependent on implementation, the electrical conduits 144 may comprise physical wiring, silicon wafers, metal tracings, and/or any other type of durable electrically conducting material.

Masking and sputtering may be used to create a connecting hole and lead/connector sites directly onto the antenna. The antenna may be attached to the PEEK implant body 110 using adhesives, or with other bonding techniques. For example, in some variations, laser welding is used to adhere the antenna wire to the PEEK surface. This may facilitate connection to a sputtered electrical conduit 144 layer within the implant body 110. Electrical conduits 144 may travel straight along, the implant body 110, or it may be threaded through the implant body. In another variation, the electrical conduits 144 (e.g., conductive metal traces) may be at least partially etched, sputtered, or embedded on the surface of the of the surgical implant (for example by welding metal foil onto the surface, by sputtering, or other methods). In another variation, the electrode circuitry may comprise conductive traces on the internal surface of a tubular (or open-section) of the surgical implant (e.g., along the tubular section of a surgical nail.

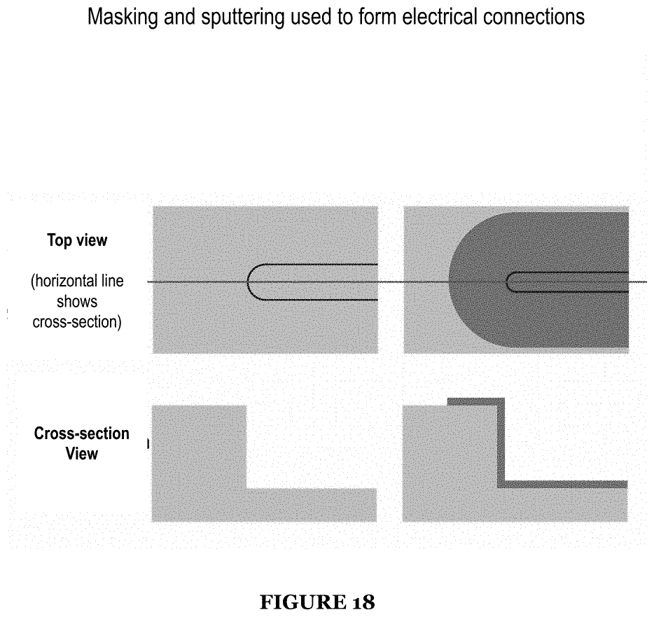

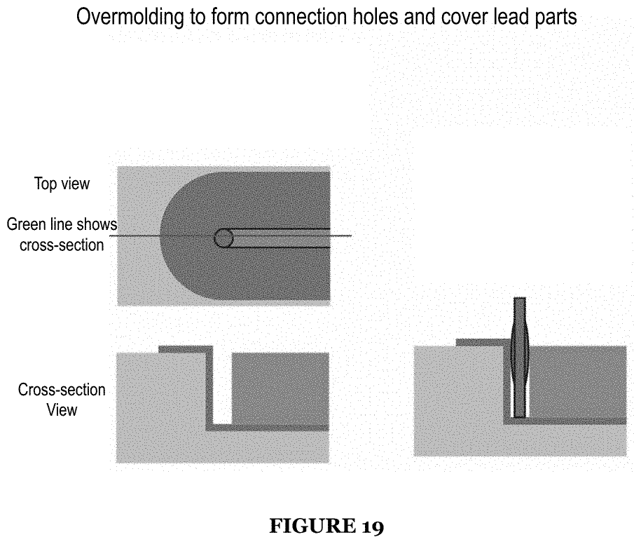

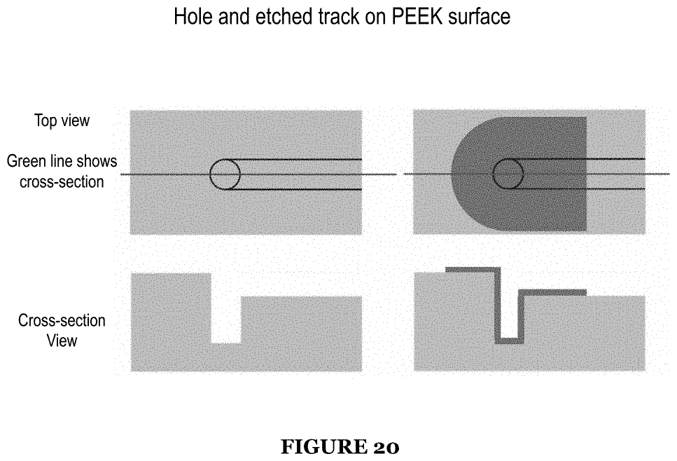

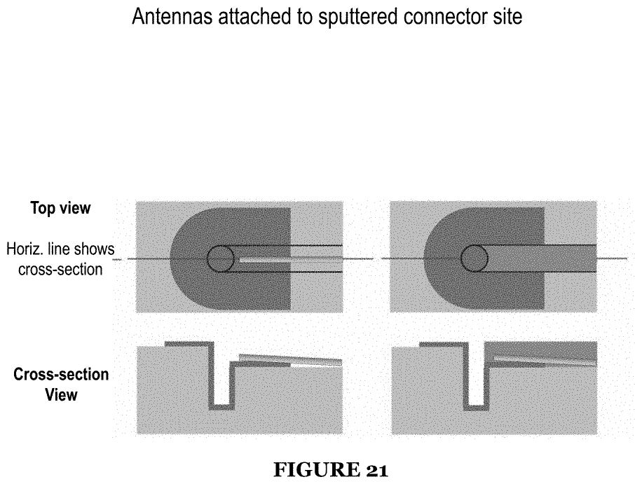

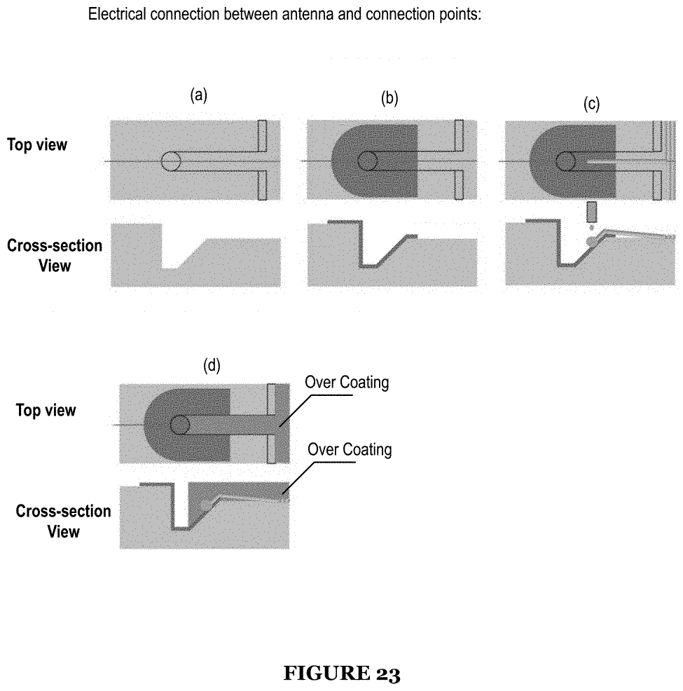

It may be required to cover/protect electrical conduits 144. General wiring may already come with insulation. As shown in FIGS. 18-21, electrical conduits 144 may additionally, or alternatively, be covered/protected by embedding them in implant body 110, either by tunneling through the body or overmolding the circuitry in an over-coating 114 layer. Other variations are also shown in FIGS. 23-25.

As shown in FIG. 23, (a) a hole or etched track can be made onto a surface (e.g., a PEEK surface of an inner frame 112); (b) an antenna wire can be connected to a connector point by extending a wire to a conductive layer (e.g., sputtered metal on connector point); (c) optionally, epoxy may be used to create a mechanical and/or electrical connection between antenna and the conductive surface; and (d) then in some embodiments, these connection may further have one or more over-coatings 114 (d). Overmolded material may further mechanically stabilize the connection and may ensure that the epoxy is not in direct contact with an outer surface of the device.

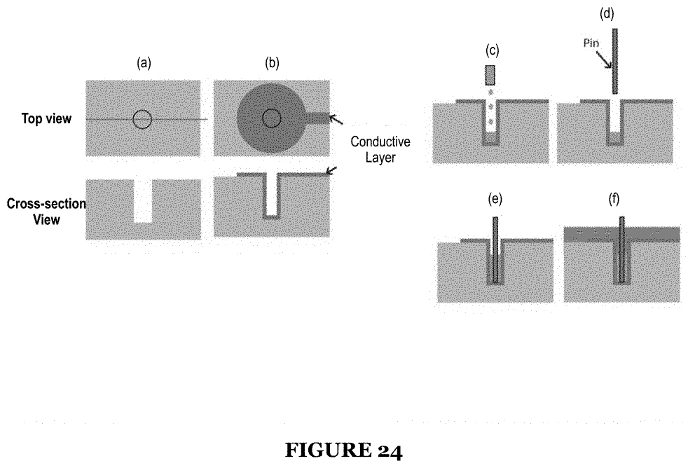

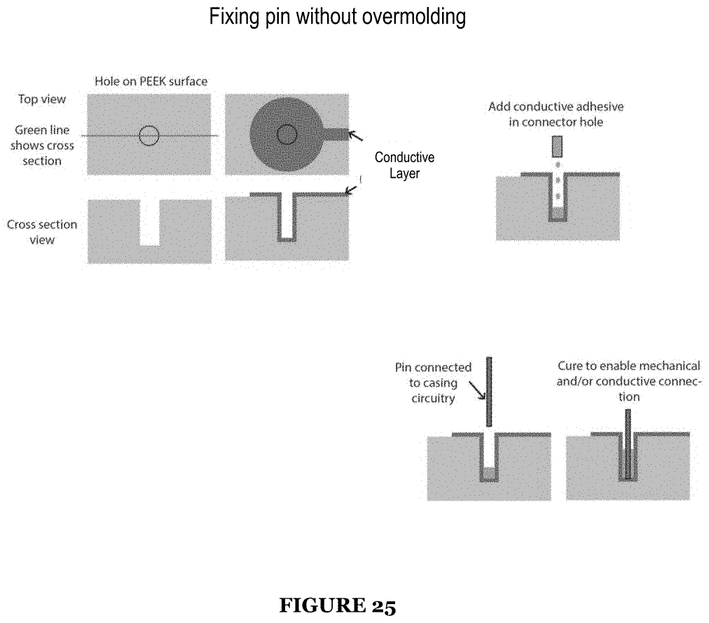

A connector pin may be integrated with a conductive path. As shown in FIG. 24, (a) a defined connector hole on an inner frame 112 or other surface (e.g., a PEEK surface) can be formed; (b) masking, sputtering or other techniques may be used to create a connector hole by establishing a conductive layer over and into the defined connector hole; (c) optionally conductive adhesive can be added to the connector hole; (d) a connector pin can be inserted into the defined connector hole; (e) the adhesive can be allowed to cure; and (f) an over coat can be overmolded or otherwise deposited as a covering. The conductive layer can go to implant body circuitry (e.g., electrodes 142 or an antenna). The adhesive may mechanically hold a connector pin and may additionally establish conductive paths between connector pins and the conductive layers. Over molding may further mechanically fix the pin and additionally the adhesive can be prevented from being in direct contact with an outer surface of the device. As shown in FIG. 25, some variations may not include overmolding.

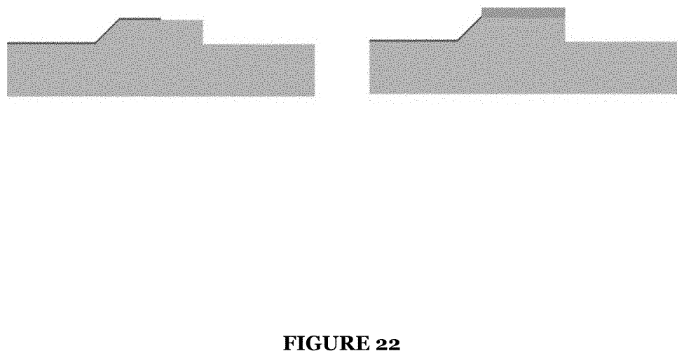

Placement of the set of conductive paths 140 may be done using many different methods. In one variation, as shown in FIG. 22 (left), a thin layer of metal is sputtered onto the electrode site (to form the electrode 142), and then extended from the electrode site along a ramp down and then along the inner frame 112 (forming an electrical conduit 144). In another variation, as shown in FIG. 22 (right), a metal foil electrode 142 is welded onto the stimulation site and conductively coupled to a sputtered layer electrical conduit layer 144. In another variation, electrode 142 and electrical conduit 144 may comprise a wire, wherein the wire is drawn along an etching within the inner frame 112 and up to the stimulation site. In this variation, the end of the wire is exposed and the electrode 142 is formed using sputtering.

As used herein, a spinal cage example will be presented for the embodiment where the inner frame 112 is composed primarily of non-conductive material. As spinal cages may be highly specialized for each individual implementation, the provided spinal cage specifications are provided as typical descriptions of that spinal cage and not presented as a limitation for the spinal cage, or the system in general.

The spinal cage variation of the inner frame 112 may be composed of any non-conductive material. In many variations, the spinal cage is composed of a polymer, such as PEEK. Alternatively, it may be made of engineered natural or synthetic bone material, or some other material. The spinal cage generally has an extruded prism geometry with many variations dependent on the specific type of spinal cage. As per a prism, the spinal cage geometry has an external surface comprising: a sufficiently, flat and opposing (e.g., parallel), top and bottom surface; and a more complex outer wall geometry that may be distinct to the specific spinal cage implementation.

The spinal cage may function as a backbone implant, implanted within, along, or in-between backbone segments to aid in connecting and enabling bone tissue growth for patient with a vertebral injury (e.g., broken vertebral plate). Once implanted, the spinal cage implant may connect two vertebral segments and enable fusion of the two segments by electrical stimulation to induce bone growth. The spinal cage may additionally include other design features such as: surface coatings (e.g., to protect the implant, increase osteo-integration, etc.), surgery tool attachment points (e.g., for easier tool utilization), teeth (e.g. to increase the chance that the spinal cage does not move), lateral openings in the spinal cage (e.g., to enable electric charge to more easily enter or exit the spinal cage and/or other elements).

The exterior perimeter of the spinal cage is defined as the perimeter along the lateral (i.e. side) wall geometry. The spinal cage can include one or more graft windows, which can be defined as internal implant cavities, wherein these internal implant cavities are defined by the interior surface of the spinal cage. Implant cavities are typically defined to be prism shaped with openings in the top and bottom of the spinal cage, which often functions to provide a through hole within which bone growth can occur. The interior surface of the spinal cage thus refers to the lateral walls that define the internal cavities. In some variations, internal cavities may have openings in addition to the top and bottom openings. As desired by implementation, these additional surfaces may also be included as part of the interior surface.

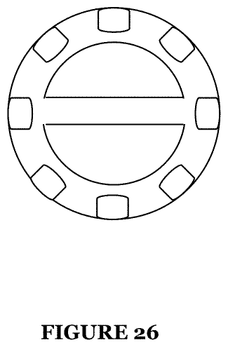

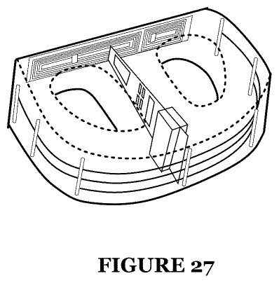

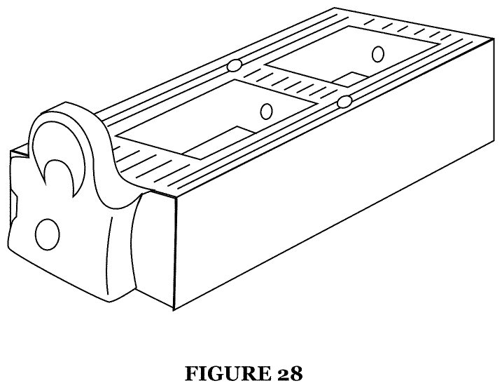

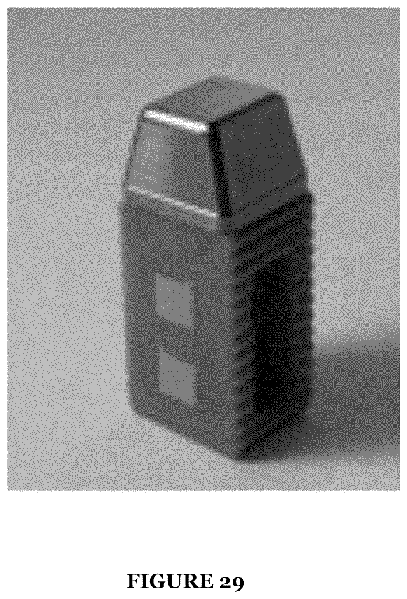

The spinal cage may be incorporated with many geometries including, but not limited to, anterior lumbar interbody fusion (ALIF) cages, transforaminal lumbar interbody fusion (TLIF) cages, posterior lumbar interbody fusion (PLIF) cages, anterior cervical fusion (ACF) cages, lateral cages and/or other suitable types of spinal cages. The spinal cage may include other design features such as: surface coatings, surgery tool attachment points, teeth, and/or other elements. Example spinal cages are shown in FIGS. 26-28. The example, FIG. 29, which shows an example TLIF cage, will be used to provide an example of the non-conductive system embodiment, but the system may be incorporated using any spinal cage, or more generally, any non-conductive surgical implant.

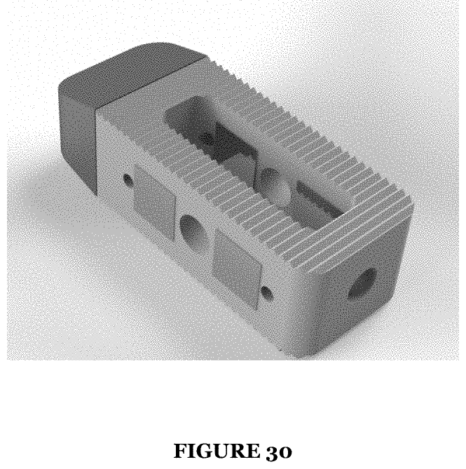

As shown in example FIGS. 29 and 30, a spinal cage will be presented with a single internal cavity graft window, extending the entire height of the implant body 110 from the bottom to the top; with four electrode sites on the exterior sides of the implant body (two on each external lateral side); four electrode sites on the interior sides of the implant body (two on each internal lateral side); and a circuitry casing 120 attached to one end of the implant body 110. Dependent on implementation of this spinal cage, the system may vary with the positioning and number of electrode sites situated throughout the implant body 110.

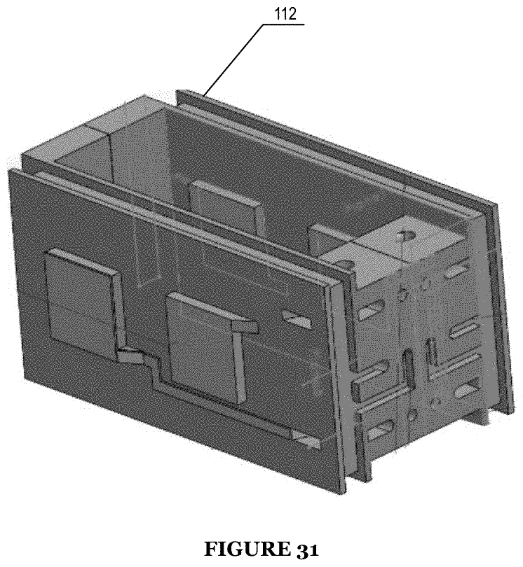

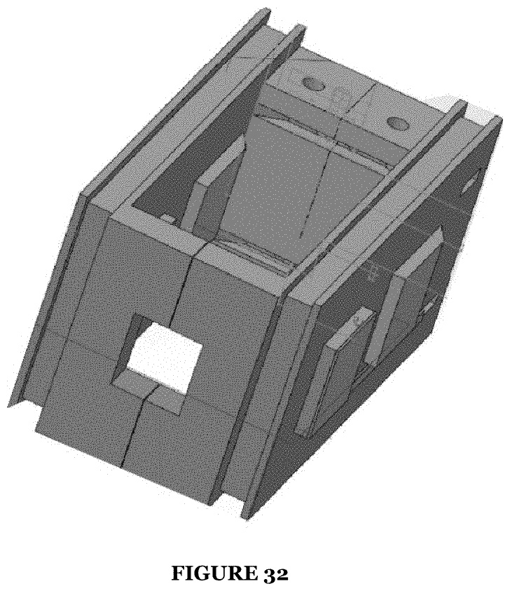

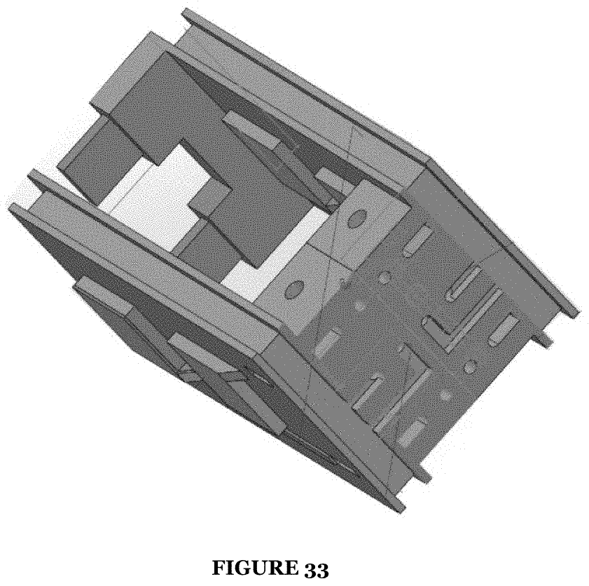

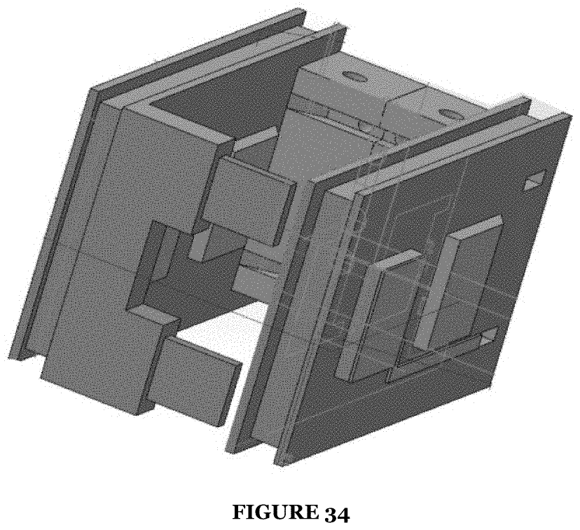

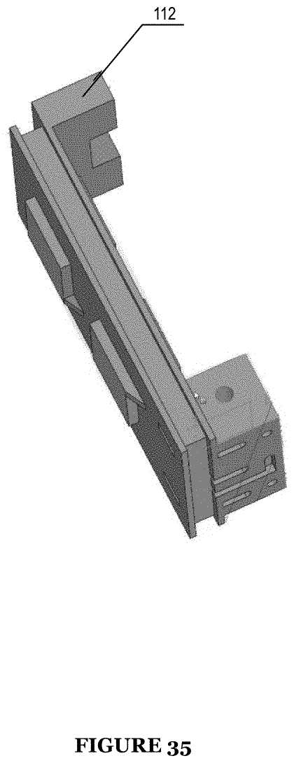

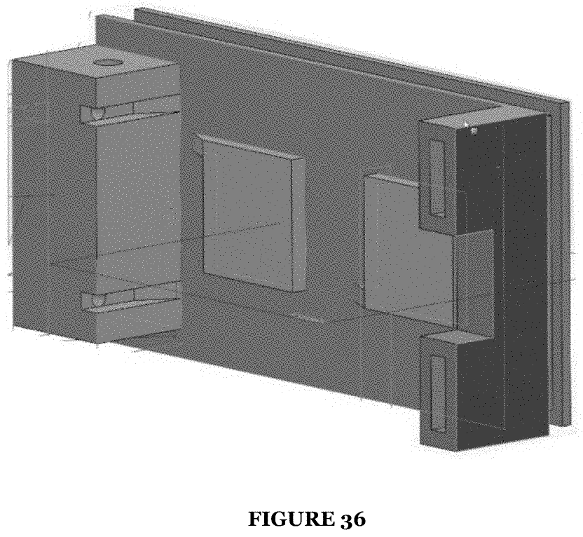

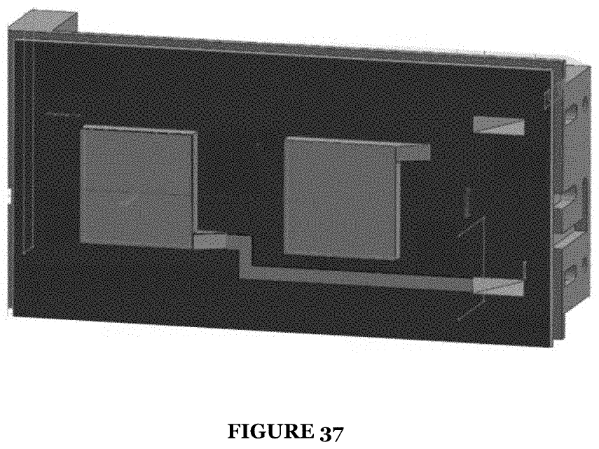

As shown in FIGS. 31-38, in this example the inner frame 112 of the spinal cage may comprise two side panels that, when combined together, form the main structure of the spinal cage. In some variations, the two side panels may be identical. Alternatively, the two side panels may have complementary components such that they connect to each other. For example, as shown in FIGS. 33 and 34, one side panel may have an extending lip piece that slides into other panel, thereby connecting the two side panels. In some implementations, the lip piece may include a “locking” protrusion enabling that lip piece to slide in and be locked in place.

For spinal cage variations, electrode sites may be situated along the interior and/or exterior lateral surfaces of the spinal cage. As shown in the example spinal cage, the electrode sites may comprise raised platforms on the external and interior surface of the inner frame 112. In this example, each side panel as four relatively square electrode sites: two raised platforms on the exterior facing side of the side panel, for electrodes 142 that can provide stimulation to the exterior of the spinal cage; and two raised platforms on the interior facing side of the side panel, for electrodes that can provide stimulation into the graft window of the spinal cage.

In addition to including electrode sites, the inner frame 112 may further include a shaped out pathway for the rest of each conductive path (e.g., molded or etched pathway). That is, the inner frame 112 may include a shaped pathway to enable positioning of electrical conduits 144 along the inner frame. As shown in the spinal cage example, a ramp may be connected to each electrode site. Additionally, dependent on implementation, an indentation (or recessed surface) within the inner frame 112 may travel along the inner frame. The ramp may enable connection of an electrical conduit 144 traveling along the inner from 110 to the electrode 142 positioned at the electrode site. Additionally, the recessed surface within the inner frame 112 may enable the electrical conduit 144 to travel along the inner frame. In some variations, particularly dependent on how the electrical conduits 144 are added, an additional indentation in the inner frame 112 may not be required. For example, if the electrical conduits 144 are sputtered (e.g., metal film sputtering) onto the inner frame 112, an indentation into the inner frame may not be required but may be beneficial during overmolding. In another example, where the electrical conduits 144 comprise wires, the indentation may be required for positioning of the wires. In various variations, indentations may be filled with a sealant/adhesive prior to overmolding to cover traces/wires/foil within the indention which may protect the trace/wire/foil against exposure/forces exerted by the high pressure polymer during the overmolding process.

In some variations for the spinal cage inner frame 112, the side panels may include an antenna housing. The antenna housing may comprise a groove, or lowered region, along the perimeter of the rectangular part of the side panel. The antenna housing may function to hold an antenna coil, as shown in FIGS. 16 and 17.

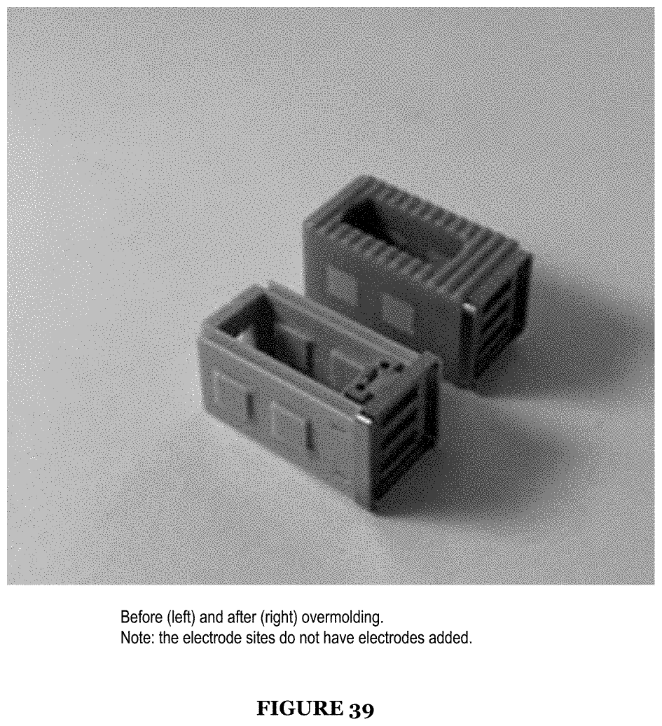

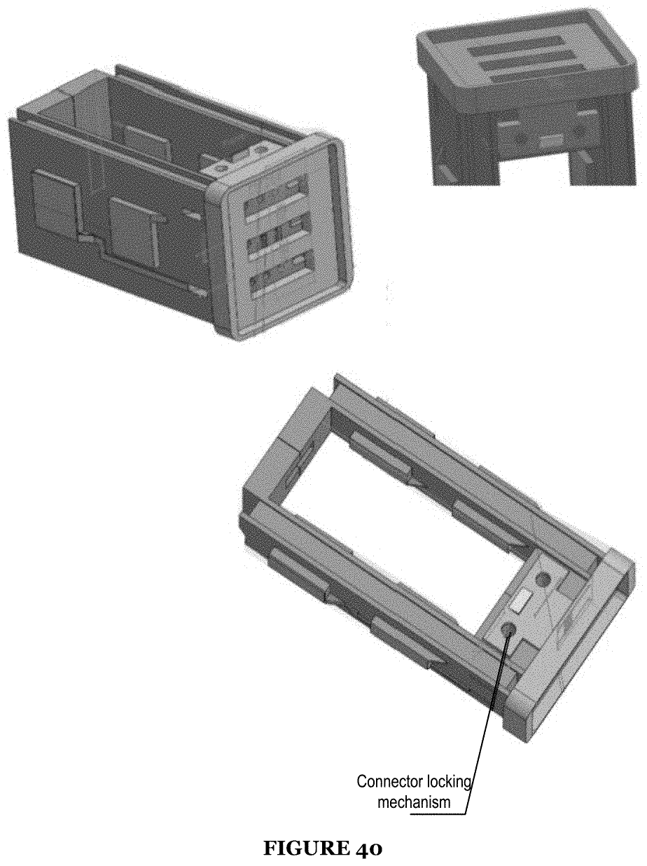

In many spinal cage variations, the inner frame 112 may include additional connector pieces to assist in connecting the electrical casing 120 to the implant body 110. The connector piece may function to enable multiple methods of attachment. As shown in FIGS. 39-41, the connector piece may comprise an adjoining piece that fits over one end of the adjoined side panels. That is, once the side panels are attached together, the connector piece may fit and/or lock into place (e.g., by overmolding) on the end that will connect to the circuitry casing 120. The circuitry casing 120 may then be adjoined to the connector piece (e.g., by welding).

In some variations, the inner frame 112 may have holes to enable the set of conductive paths 140 to pass on and through the implant body 110. These holes may enable electrical conduits 144 to travel between the interior and exterior of the implant body 110, help enable electrical connections between the implant circuitry 130 within the circuitry casing 120 and implant circuitry in/on the implant body (e.g., enable connection of an antenna on the inner frame 112 with the implant circuitry), and help enable electrical connections between the implant circuitry and the set of conductive paths 140. The connector piece may contain holes, and/or slits enabling electrical conduits 144 to pass through the connector piece to the electrical casing 120. In many variations, the connector piece is composed out of PEEK or other non-conductive material. Alternatively, the connector piece may be composed of metal (e.g., titanium).

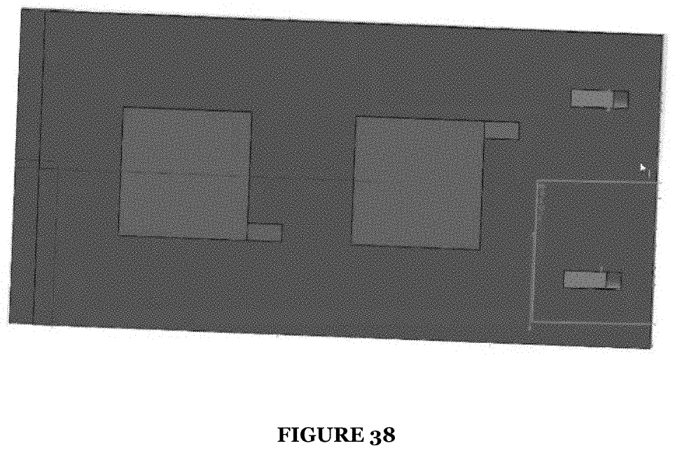

As shown in example FIG. 38, the side panels of the inner frame 112 may have holes such that the electrical conduit 144 attached to each electrode 142 may pass from the exterior of the side panel to the interior of the side panel (i.e., one hole for each electrode). Additionally or alternatively, in some variations, the inner frame 112 (e.g., as shown in FIGS. 31, 33, and 37) may have holes at the end of the implant body 110 that connects to the circuitry casing 120. These holes function to enable an electrical connection with the implant circuitry 130 within the circuitry casing 120.

The spinal cage variation of the system may include an over-coating 114. As a first embodiment variation, multiple layers of over-coating 114 are not required, although may be implemented as desired. In addition to providing insulation for conducting components, the the over-coating 114 may provide additional functionality. This may be particularly true for PEEK, or other plastic, overmolding over-coating 114. In these variations, an over-coating layer 114 may further help hold the inner frame 112 side panels of the spinal cage together. Additionally, the over-coating 114 may be shaped to provide other functionality. For example, as shown in FIGS. 29 and 30, the additional over-coating 114 (PEEK) layer may be grooved as teeth to aid the spinal cage in staying fixed between a patient vertebrae. FIG. 39 shows two sample prototypes of the implant body 110, one comprising just the inner frame 112 (left), and one that include over-coating 114 PEEK layer overmolded over the inner frame. Furthermore, an overmolded PEEK over-coating 114 may be used to anchor a connector piece to the PEEK side panels.

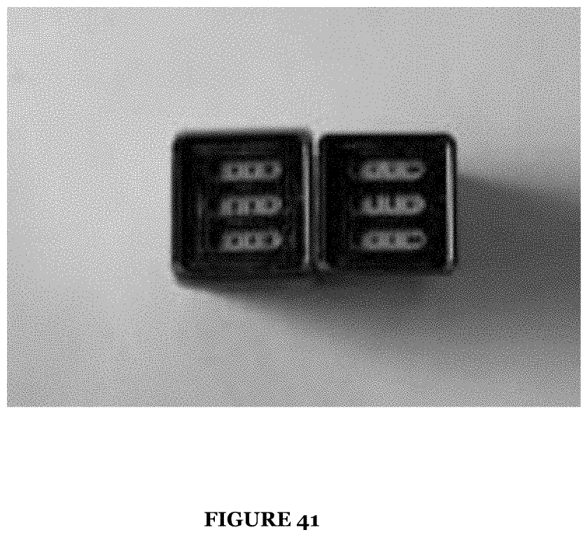

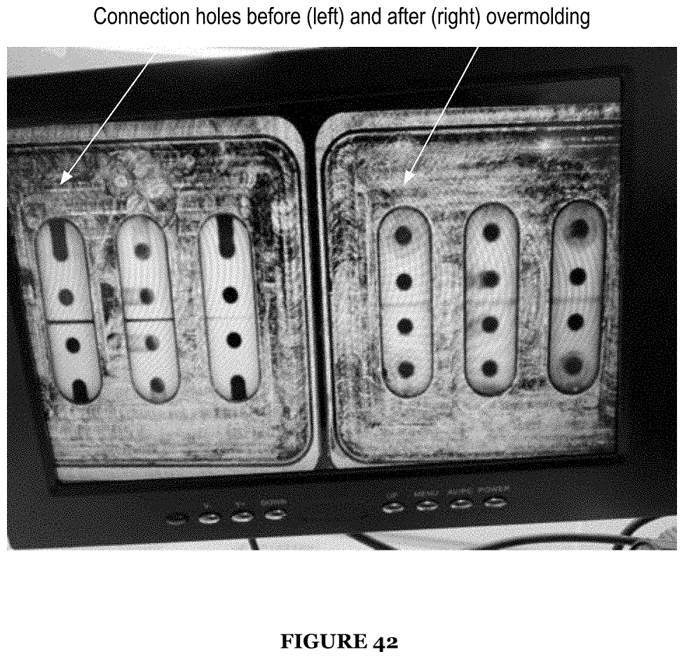

In variations where the holes in the connector piece are used as tunnels for conductive wires/traces between the set of conductive paths 140 through the implant body 110, and the implant circuitry 130 located in the circuit casing 120, the over-coating 114 may be used to completely or partially fill the remaining volume of the holes within the connector piece. This may be particularly useful when the wire/trace traveling through the holes do not completely fill the empty space of the hole cavity. Example implementations of PEEK filled holes are shown in FIGS. 41 and 42.

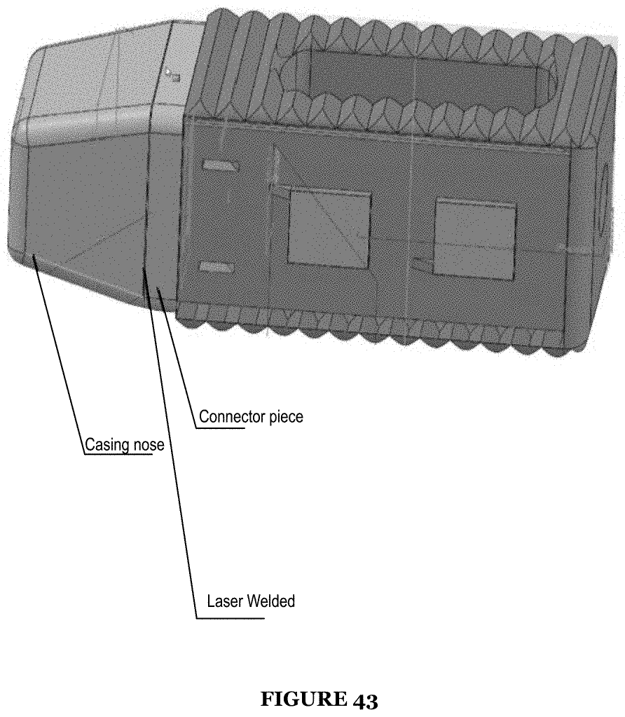

The spinal cage variation of the system may include a circuitry casing 120. The circuitry casing 120 functions as a housing for at least some of the implant circuitry 130 of the spinal cage. The As described above, the circuitry casing 120 may be mechanically and electrically connected to the implant body 110 of the spinal cage. In many variations, the circuitry casing 120 is connected to the implant body 110 via the connector piece, as shown in FIG. 43.

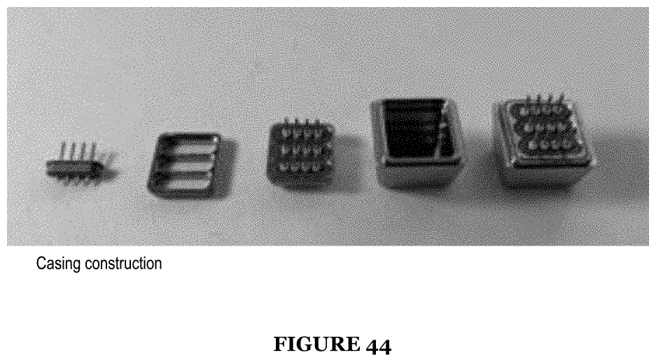

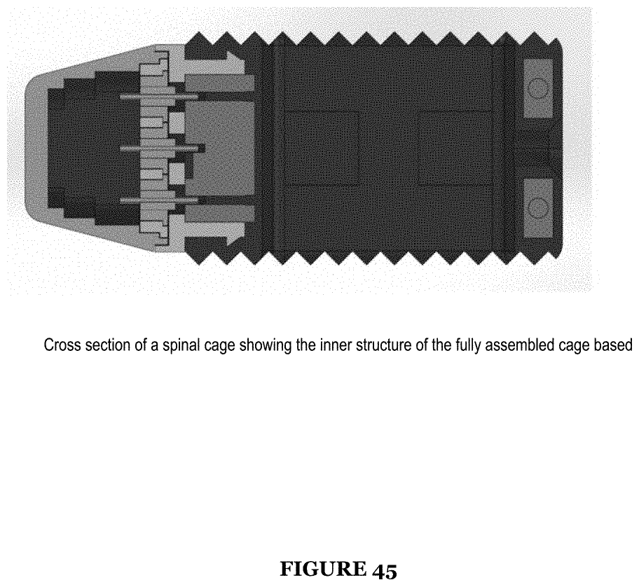

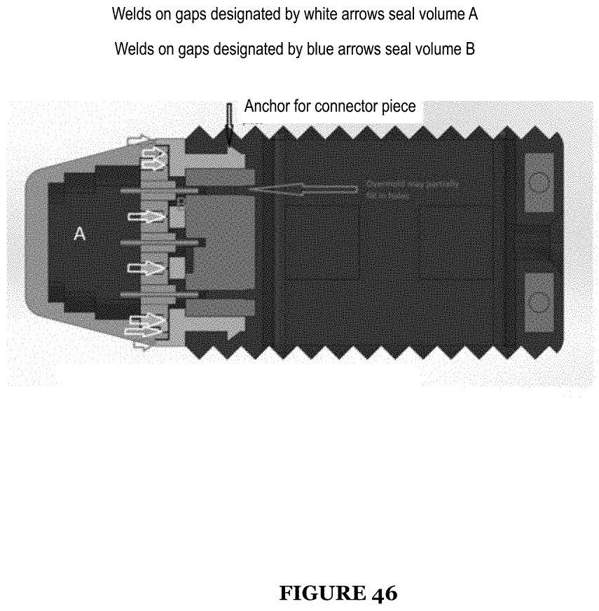

In this spinal cage variation, the circuitry casing 120 may comprise a distinct body structure “nose” on the side of the spinal cage. In some variations, the system may have multiple casings. In one sample prototype, as shown in FIG. 44, the circuitry casing 120 may comprise multiple components that fit together, both providing a housing for other components and sealing the components within. Dependent on implementation, the circuitry casing 120 may be permanently fixed in place or further detachable from the implant body 110. As a housing for implant circuitry 130, the circuitry casing 120 may be sealed, as shown for example in FIGS. 45-46, such that biological material does not flow into the circuitry casing 120 and any type of electronic residue (e.g., battery solution) does not leak out. In some variations, the casing may be hermetically sealed.

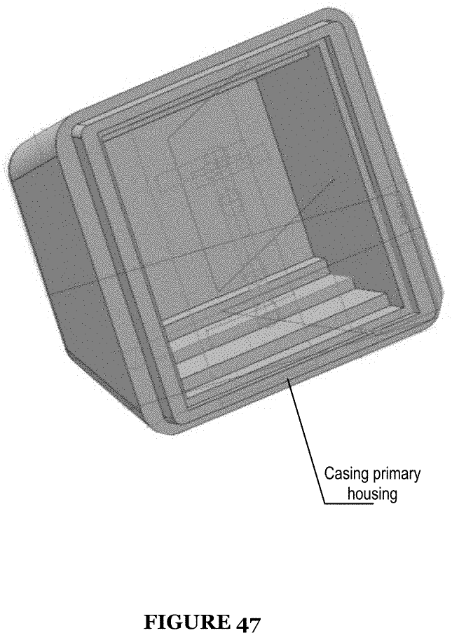

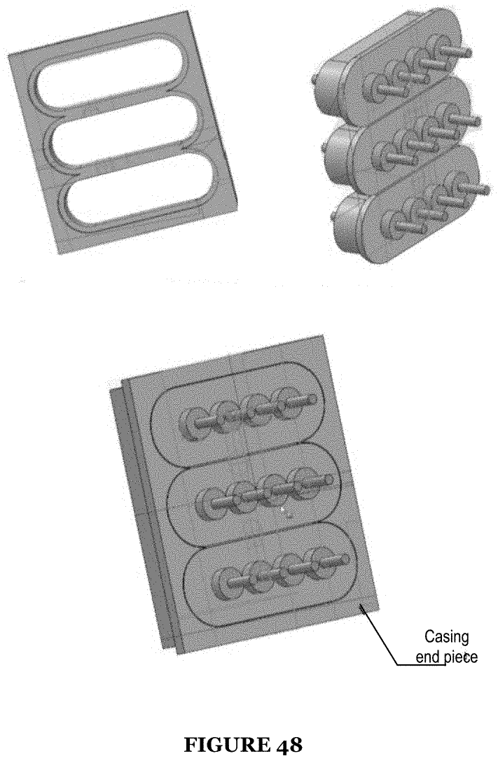

In this example for a titanium circuitry casing 120 for a TLIF cage spinal cage, as shown in the design schematic of FIG. 43, the casing may be positioned on the lateral exterior of the implant body 110; along a shorter side of the implant body 110. As shown in FIGS. 47-49, the circuitry casing 120 may comprise two pieces: a primary housing, containing the implant circuitry 130; and an end piece containing wiring (e.g., feedthroughs) that connects to the set of conductive paths 140 (and possibly other electrical components such as antennas) within the implant body 110. The wiring through the end piece may be sealed such that no air passes through the wiring holes (e.g., hermetic feedthroughs).

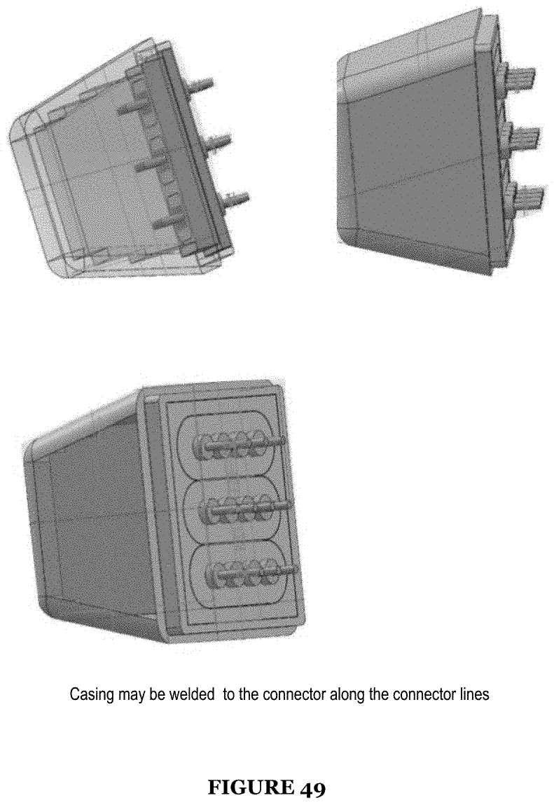

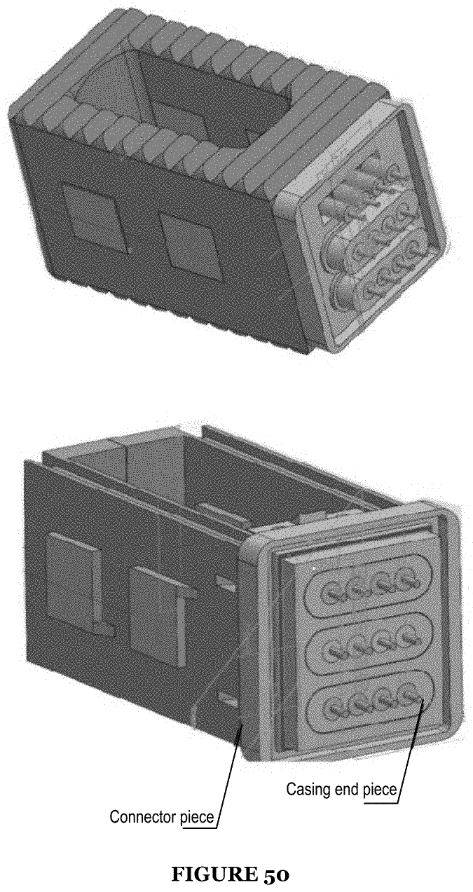

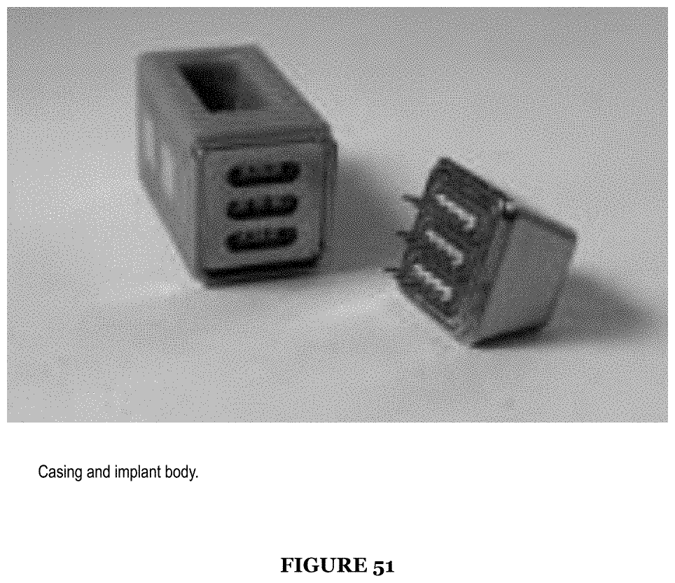

As shown in FIG. 49, the connector piece may be fitted to sit on the shorter, “nose” end of the spinal cage. The connector piece may have a locking mechanism (e.g., locking rod, locking shapes, and/or anchors etc.) that would lock the connector piece onto the implant body 110. Dependent on manufacturing implementation, the circuitry casing 120 end piece, may be first sealed to the connector piece (as shown in FIG. 50) prior to attachment of the primary housing. Alternatively, the circuitry casing 120 may be initially sealed prior to attaching it to the connector piece. In some variations, the casing may be hermetically sealed (e.g., by welding) to the connector piece. One example prototype of the implant body 110 and the circuitry casing 120 are shown in FIG. 51.

The spinal cage variation of the system may include implant circuitry 130 comprising implant receiver circuitry 132, implant control circuitry 134, and a power system 136. The majority of the implant circuitry 130 may be fitted on a PCB within the circuitry casing 120 as described previously. In some variations of the implant receiver may include one, or two, antennas outside of the circuitry casing 120. As shown in FIG. 1 at least one antenna may be located on around part of the inner frame 112. In one example, the antennas may be wired loops around the perimeter of the longer faces (e.g., lateral faces) of the side panel. The antenna may then be electrically connected to other implant circuitry 130 using the electrical conduits 144 (or similar conduits designed for the antennas). In another variation, the antenna may be printed, or attached, onto a circuitry surface (e.g., IC or a PCB). As an antenna is more flexible, the antenna may, or may not, be positioned on a bent part of the circuitry surface. In one example, the antenna may comprise multiple antennas that span three-dimensional space. In this example, the PCB may be folded such that the PCB has at least one normal surface to all three space dimensions. An antenna may then be printed on each normal surface of the PCB such that the antenna spans all space dimensions. Generally, the PCB may have any number of folds, wherein all folds combined, at least, span all space dimensions. Antennas may then be mounted on sufficient PCB surfaces such that the antenna spans all space dimensions. In a third variation, the antenna may comprise connected, or disjoint, structures along folds of a PCB folded in an accordion manner. That is, the antenna may leverage the PCB folding to create an extended antenna, wherein loops (or other another geometric structure) of the antenna are linearly positioned along each segment of the fold.

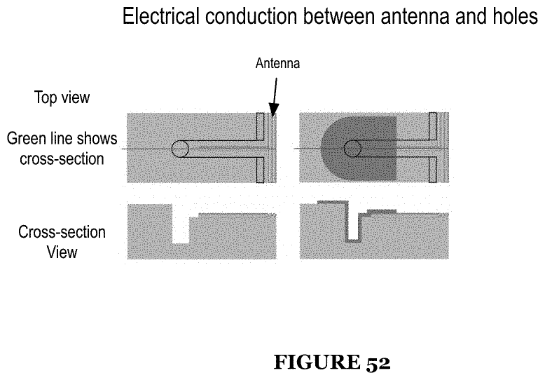

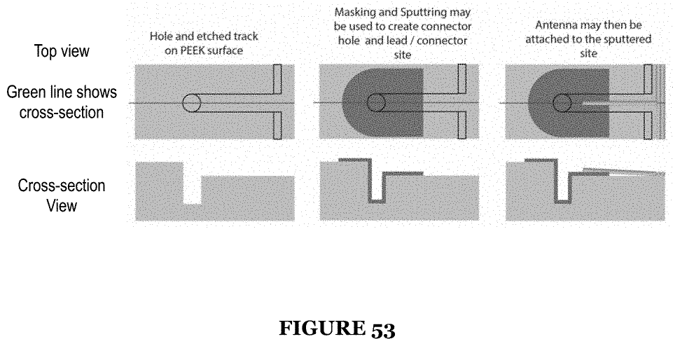

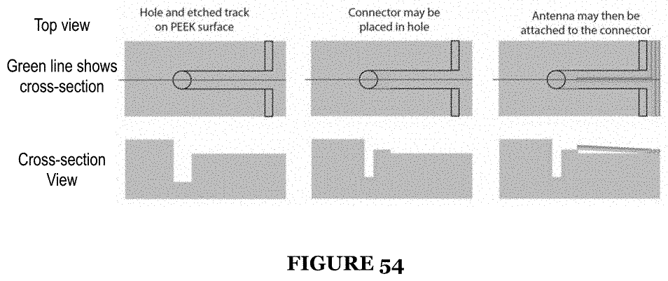

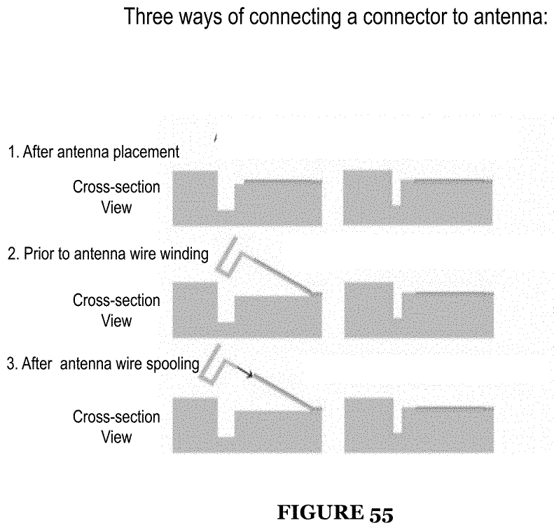

Electrical conduits 144 may connect the implant body antenna to the implant circuitry 130. As shown in one example schematic of FIGS. 52-55 and sample prototypes FIGS. 50 and 51, etched tracks may create antenna conduits between holes in the side panels or connector piece to the antenna. Masking and sputtering may be used to create a connecting hole and lead/connector sites directly onto the antenna. The antenna may be attached to the PEEK implant body 110 using adhesives, or with other bonding techniques. For example, in some variations, laser welding is used to adhere the antenna wire to the PEEK surface. This may facilitate connection to a sputtered electrical circuitry layer.

As used herein, a surgical nail (also called intermedullary rod) will be presented for the embodiment where the inner frame 112 is composed primarily of conductive material. As surgical nails may be highly specialized for each individual implementation, the provided specifications are provided as typical descriptions for that surgical nail and not presented as a limitation for the surgical nail, or the system in general.

The surgical nail (also referred to as intermedullary rod or intermedullary nail), may be of any typical, or non-typical shape or size dependent on the required implementation. For example, the surgical nail may be straight, bent, solid, hollow, include openings, etc. The surgical nail comprises the shaft of the orthopedic implant, wherein one end connects to the end cap (i.e., the head) and the opposite end comprises the tail end and the elongated portion extending from the head to the tail is described as the “length” of the nail. As a surgical nail, the tail end may have an equal or lesser cross-sectional area as compared to the head end. One example illustration of the surgical nail. The surgical nail may have any common, or uncommon, attachments, such as screws and fasteners.

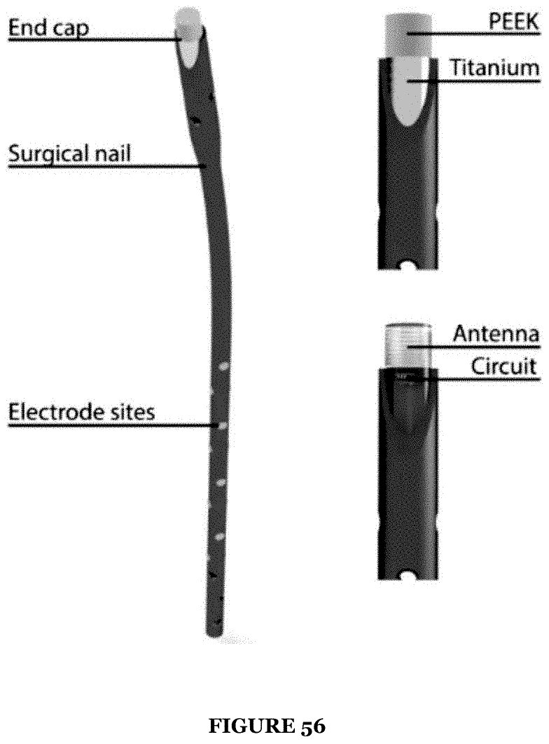

As described above, and shown in examples FIGS. 3 and 4 and prototype example FIG. 56, the surgical nail system variation may comprise: an implant body 110, that includes a cylindrical inner frame 112, and an over-coating 114 that covers all electrical components; an circuitry casing 120, connected at one end of the implant body (called the head region), wherein the circuitry casing 120 contains implant circuitry 130; which are electrically connected to a set of conductive paths 140 that travel along the length of the surgical nail (i.e., electrical conduits 144) and provide stimulation at exposed regions (electrodes 142) on the implant body 110.

The surgical nail inner frame 112 primarily comprises a single cylindrical body (i.e., shaft) wherein dependent on implementation, the inner frame 112 of the surgical nail is preferably constructed of a durable non-toxic, minimally corrosive, material. In some variations, the surgical nail is composed of titanium. Additionally or alternatively, the surgical nail may include other non-toxic metals, or metal alloys, such as: titanium alloy, platinum, stainless steel, cobalt-chromium alloys, tantalum, and/or any combination of thereof. The surgical nail may additionally or alternatively, be at least partially composed of non-metallic compounds, such as: biomedical tissue, silicone, or plastics (e.g., polyether ether ketone (PEEK)).

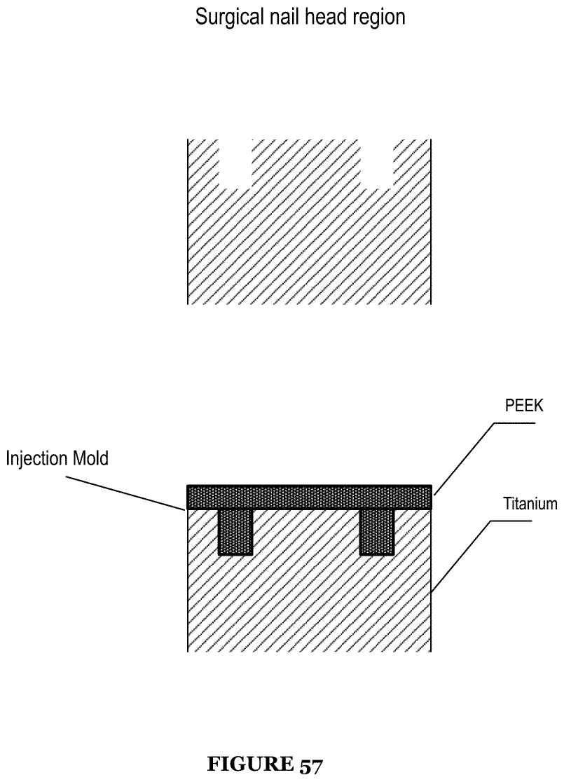

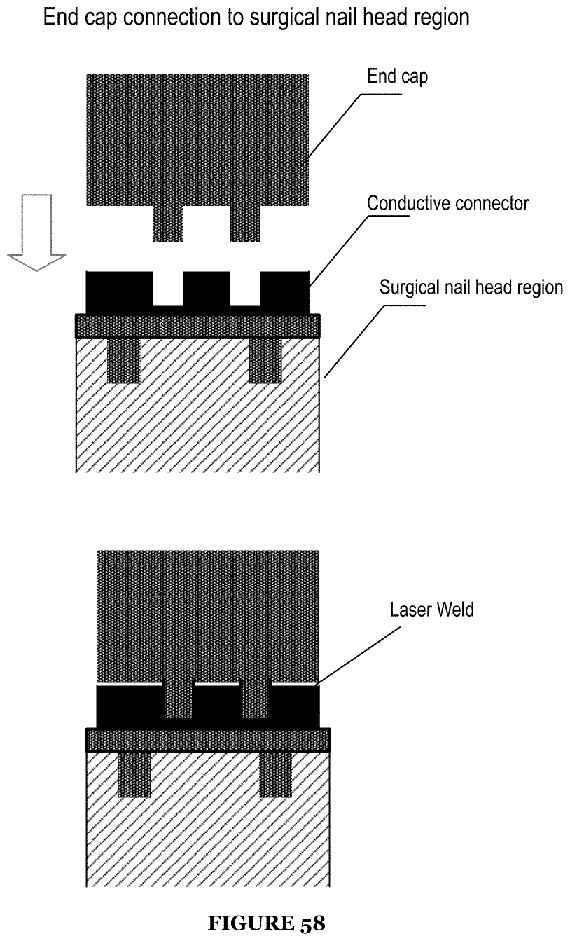

The head (or head region) of the inner frame 112 may comprise an opening cavity that enables attachment of the circuitry casing 120. Alternatively, the head region of the inner frame 112 may not include an opening cavity. The head region may be composed of the same material as the rest of the surgical nail (e.g., titanium), or may be composed of different materials (e.g., PEEK) or combinations of different materials. For example, as shown in FIGS. 57-58. The head region may be partially composed of titanium and PEEK, wherein the PEEK region includes extensions into the titanium region, thereby fastening the PEEK into place (e.g., through injection molding). Alternatively, the titanium region may extend into the PEEK region. In some variations, the head region cavity may be threaded to enable the circuitry casing 120 to be “screwed on”. Other attachment mechanisms (e.g., mechanical fastener) may alternatively be used to enable an circuitry casing 120 to be attached to the head region to mechanically and conductively couple the circuitry casing to the surgical nail. Additionally or alternatively, the head region may be lined by some material to provide a seal with the end cap (e.g., PTFE tape, or a washer). Additionally or alternatively, the head region may be composed of a material that enables the use of an adhesive to fix the circuitry casing 120 in place.

The inner frame body, i.e., the shaft region, may be an elongated shape. The body serves as the primary volume of the orthopedic implant. The surgical nail body may be straight, curved or elongated along any suitable path. In some variations, the surgical nail body may include holes approximately orthogonal to the length of the surgical nail. These holes may function to enable screws or other components to fix the surgical nail in place.

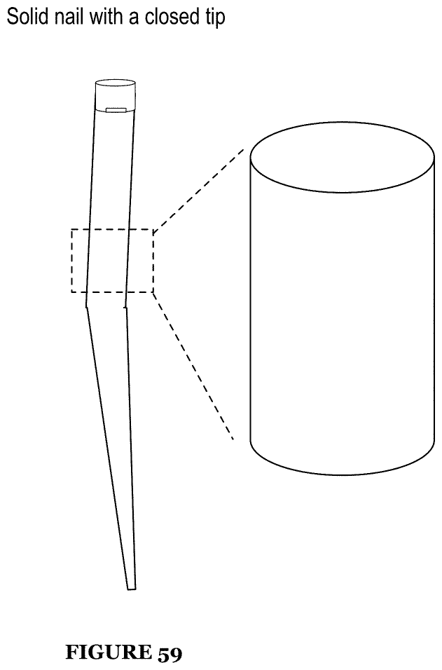

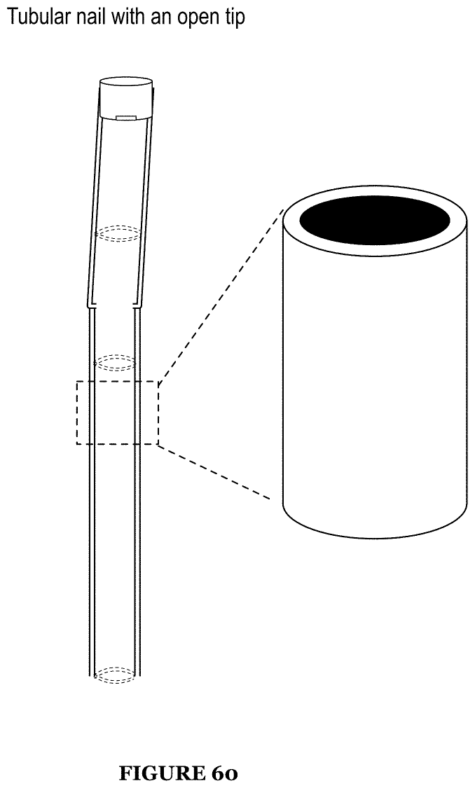

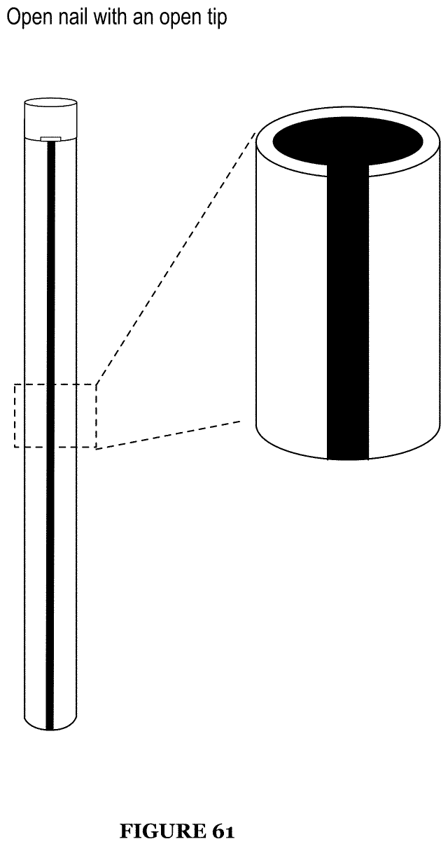

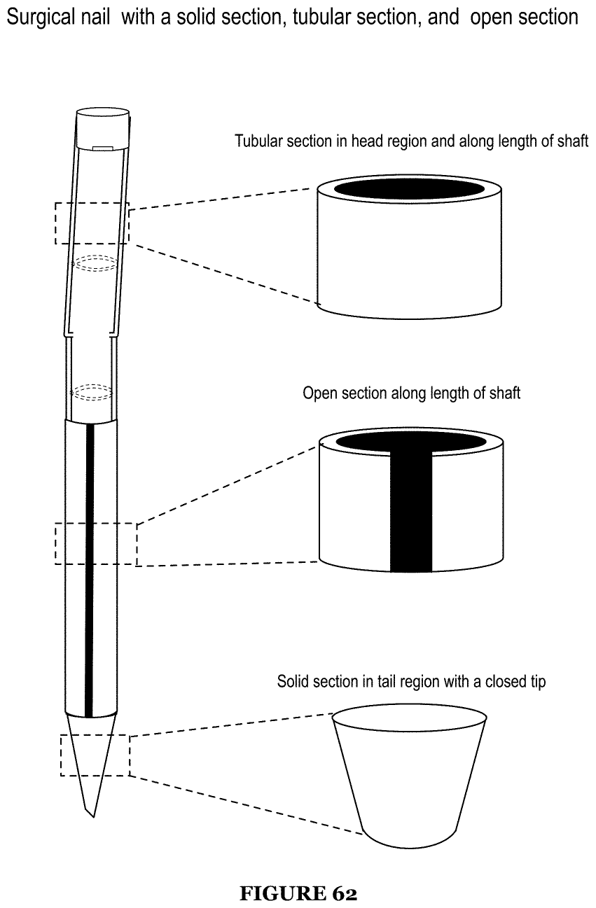

Dependent on implementation, the surgical nail body may be solid, as shown in example FIG. 59; tubular (i.e., include an internal cavity), as shown in example FIG. 60; be open, as shown in example FIG. 61; and/or some combination of the three, as shown in example FIG. 62. That is, dependent on implementation the shaft of the surgical nail may be: entirely solid, tubular, or open; or comprise any combination of solid, tubular, and/or open sections. Independent of the shaft composition, the tail may be open or closed.

The shaft of a tubular nail or a tubular section may be substantially hollow, wherein the shaft includes a defined internal cavity extending along the tubular section of the nail. In many variations, the proximal end, i.e., head, comprises an opening of the internal cavity. Dependent on implementation, the internal cavity may also have an opening at the distal end. This opening may be as large, or smaller than the opening at the head. The size (i.e., cavity diameter, or cross-sectional area) of the cavity may vary dependent on implementation. In some variations, the cavity may comprise a significant cross-sectional area of the surgical nail. In other variations the cavity may be significantly smaller (e.g., just sufficient to enable wire to pass through the interior of the surgical nail.

The shaft of an open nail or an open section of a nail may also be substantially hollow, wherein the shaft includes a defined internal cavity. Additionally, open segments include an “opening” such that along the open segment of the nail, the interior surface and the exterior surface form a continuous surface. In the example FIGS. 61 and 62, the opening is shown as a slit along the length of the nail, but generally, the opening may have any desired shape dependent on implementation. For example, in variations, where it is desired that biological mass eventually envelops the surgical nail, the open section may span the entire nail to enable biological material to completely grow into the nail. In another example, the open sections may comprise small holes enabling electrodes 142 within the nail to be exposed on the exterior of the surgical nail.

For surgical nail variations, the shaft may have electrode sites Additionally or alternatively, the head and the tail of the surgical nail may also include electrode sites. The shape and positioning of the electrode sites may vary dependent on implementation. For example, on solid regions of the surgical nail, electrode sites may comprise “etchings” on the exterior surface of the shaft, such that an electrode 142 may be fitted into (or onto) the etched region(s). In tubular sections of the shaft, electrode sites may be fitted in holes (or other openings) along the body of the shaft such that electrical conduits 144 may travel through the tubular region with the electrodes 142 situated exposed on the exterior surface of the shaft. Holes, or open sections, may also be used to aid in connecting wiring traveling though the tubular region to electrodes sites exposed on the exterior surface of the shaft. For open-sections of the of the shaft, electrode sites may be situated anywhere within (along the open-section), or along the shaft.

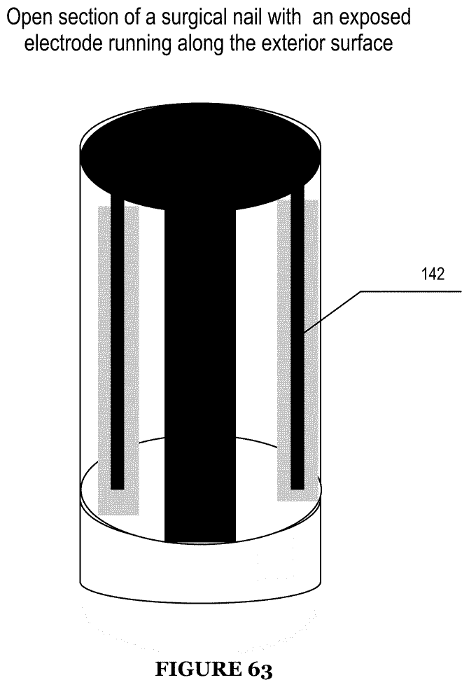

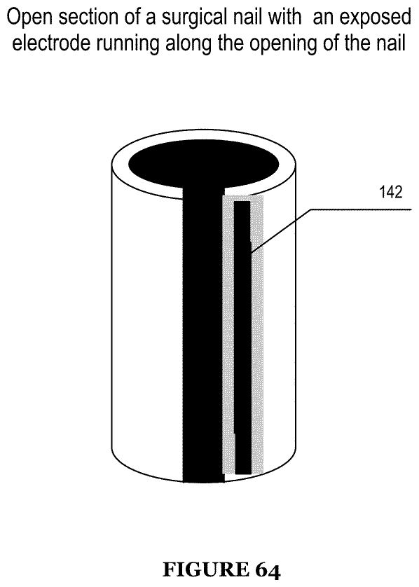

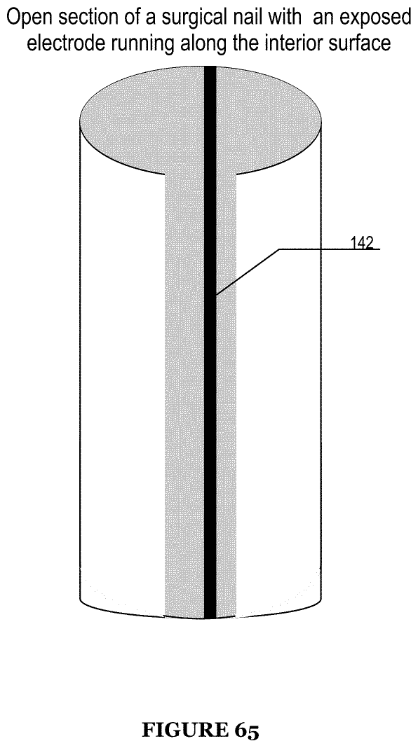

For example, in one open-section implementation, one electrode site may comprise the entire length of the open section. In different implementations, electrode sites may be along the exterior surface of the open section, along the opening of the open section, on the interior of the open section but sufficiently exposed to the exterior, or any combination thereof. In one example, as shown in FIG. 63, the electrode site extends along the exterior surface of the surgical nail. In another example, as shown in FIG. 64, the electrode site extends along the opening of the surgical nail. In another example, as shown in FIG. 65, the electrode site extends along the interior surface of the open section. Although shown directly opposite the opening, the electrode site may be anywhere along the interior surface and may be positioned such that it is sufficiently close to the opening of the open section to affect an electric field on the exterior of the surgical nail. Dependent on implementation, exposed electrode sites may be situated on, or have an insulation covering or layer to separate conductive components (e.g., separating the orthopedic implant 110, and each electrode 142). This insulation covering (discussed further below) may be implementation specific and dependent on the implant composition, electrode 142 positioning, and desired type(s) of stimulation.

The inner frame 112 may further include a connector piece. The connector piece may be located in the head region of the implant body 110 The connector piece functions as the electrical and mechanical connector between circuitry casing 120 and the rest of the surgical nail. In this manner, the connector piece preferably includes electrical pathways (e.g., wires) that connect to the circuitry within the circuitry casing 120 and to the set of conductive paths 140 within the surgical nail. Additionally or alternatively, the connector piece may include locking mechanism(s) to enable end cap and/or surgical nail attachment. The conductive connector may be produced in a similar fashion as the end cap and may be composed of plastic and/or metal. In some variations, the connector piece is shaped such that once attached to the circuitry casing 120, the circuitry casing is sealed, preventing fluid ingress. In some variations, the connector piece is shaped such that once attached to the circuitry casing 120 and the inner frame 112, the implant circuitry 130 is electrically connected to the set of conductive paths 140 within the implant body 110. In some implementations, the connector piece furthermore forms a hermetic seal with the end cap.

The surgical nail variation, implant body 110 may include an over-coating 114 (also referred to as over-coating layer, insulation, or insulation layer). As a second embodiment variation, multiple layers of over-coating 114 may be generally required, a first over-coating 114 layer on the inner frame 112 to isolate the set of conductive paths 140 from the inner frame, and at least a second layer to isolate electrical conduits 144 from the external environment. The over-coating 114 may further provide structural support (e.g., overmolding the inner frame 112 shaft to the connector piece), and provide a desired external geometry (e.g., ridges or grooved teeth to prevent movement).

The surgical nail system variation may include a circuitry casing 120. The circuitry casing 120 functions as a housing that contains the implant circuitry 130 and other electronic components of the system. The circuitry casing 120 may connect and fasten to the head region of the surgical nail as described above. Dependent on implementation, the circuitry casing 120 may be permanently fixed in place or further detachable from the implant. As a housing for electronic components, the circuitry casing 120 may be sealed, such that biological material does not flow into the circuitry casing 120 and any type of electronic residue (e.g., battery solution) does not leak out of the circuitry casing. In some variations, the circuitry casing 120 is hermetically sealed.

The surgical nail system variation may include implant circuitry 130. The implant circuitry 130 may comprise implant receiver circuitry 132, implant control circuitry 134, and a power system 136. As described above, the implant circuitry 130 may be primary situated within the circuitry casing 120. In surgical nail variations, an antenna may be also located in the circuitry casing 120. In some examples, the antenna is isolated in a secondary housing.

The surgical nail system variation may include a set of conductive paths 140, comprising electrodes 142 situated on electrode sites, and electrical conduits 144 that extend from the electrodes along the inner frame 112 to the implant circuitry 130. Electrodes 142 function to provide electrical stimulation (e.g., for treatment). Each electrode 142, includes a distinct stimulation site and circuitry (electrical conduits 144) coupled to the implant control circuitry 134 within the circuitry casing 120.

As described above, each electrode 142 may be individually controllable. The shape, size, and number of electrodes 142 may be implementation specific. In some variations, as shown in FIG. 56, the electrodes 142 may comprise round pads on the exterior of the shaft of the surgical nail. In one example of this variation, the surgical nail may have a set of eight electrodes 142, with round pad stimulation sites, positioned around the shaft. These pads may be composed of non-toxic conductive material (e.g., titanium). In some variations, the electrode sites on the shaft may be shaped such that the electrode 142 stimulation sites lock into the electrode sites. Alternatively, electrode 142 may be molded, adhered, soldered, or otherwise attached into each electrode site (potentially with some insulation between the electrode site and the rest of the shaft). In another variation, the stimulation sites may comprise flexible/semi-flexible metal plates that are folded through the electrode sites such that they stay fixed in place. In one variation, for an open surgical nail region, the electrode site may comprise conductive plates directly exposed from the interior of the surgical nail. In another variation, the stimulation sites may include conductive etchings on the surface of the surgical nail (e.g., a conductive etch on the surface of the solid surgical nail.

The set of conductive paths 140 may include electrical conduits 144. Electrical conduits 144 functions as the electrical connection between the electrodes 142 and the implant circuitry 130. Dependent on implementation, the electrical conduits 144 may comprise physical wiring, conductive traces on a flexible or rigid circuit board, conductive paths manufactured into the surgical nail body, silicon wafers, and/or any other type of durable electrically conducting material. In variations, where the surgical nail includes tubular and/or open regions, the electrical conduits 144 may travel through the interior of the surgical nail. The electrical conduits 144 may travel straight through the implant body 110, or it may be threaded through the shaft. Additionally or alternatively, the electrical conduits 144 may be at least partially etched or embedded on the surface of the surgical nail. In another variation, the electrical conduits 144 may comprise conductive traces on the internal surface of the tubular (or open-section) of the shaft.

As mentioned above, the over-coating 114 may comprise a covering on the inner frame 112 (or electrode 142), or may include an additional material layer. In one example of a material layer implementation, as shown in FIG. 4, the surgical nail (e.g., solid, tubular, and/or open) may have material layer insulation coating (i.e., base insulation layer) on the outer surface. The set of conductive paths 140 may include electrical conduits 144 and electrodes 142 positioned onto this base over-coating 114 layer (e.g., positioned by etching, printing, sputtering, etc.). These set of conductive paths 140 may then be covered with an additional material layer over-coating 114 (i.e., outer insulation layer), wherein only the stimulation sites of the electrodes 142 would be left exposed on the exterior of the implant. The over-coating may be composed of any non-toxic, non-conductive material. In some variations, multiple forms of insulation are used. Examples include surface coatings (e.g., polyimide, epoxy, silicone, PEEK) and wire sheaths.

### 3. Method

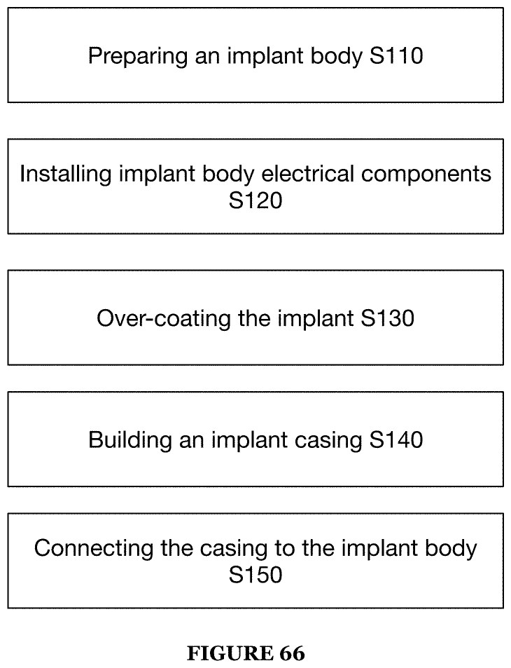

As shown in FIG. 66, a method for constructing an electrically stimulating surgical implant comprises: preparing an implant body S110, installing implant body electrical components S120, building an implant casing S130, comprising installing casing electrical components; building an implant casing S140; and connecting the casing to the implant body S150. In variations, wherein the implant body includes conductive regions, installing implant body electrical components S120 comprises: adding a base insulation layer, installing electrodes, and adding a cover insulation layer. In variations, wherein the implant body includes does not include conductive regions, installing implant body electrical components S120 comprises: installing electrodes, and adding a cover insulation layer. The method functions to construct an enhanced “electrical stimulation”-enabled implant. As part of patient treatment, this implant device may then enable improved and/or new treatment beyond the static implant device. The method may be implemented with the system as described above, but may be implemented generally as part of the construction of any implant, wherein the method may provide a surgical implant with the ability to provide controlled electrical stimulation. In this general implementation, the method may be performed in complement to an implant construction method, enabling the finished implant to have electrical stimulation, sensing, or other electronic capabilities.

The method has multiple variations that take into account the implant composition, particularly taking into account the conductivity of the implant. That is, the method has variations to take into account implants primarily constructed of conducting material (e.g., platinum and titanium), implants primarily constructed of non-conducting material (e.g., plastics and biological material), and any combination of implants with conductive and non-conductive regions. The method may also take into account unique geometries of the surgical implant, such as cavities, bends, holes, etc. That is, the method may be implemented with implants that are: entirely or partially a solid structure, entirely or partially tubular (i.e., sections of the implant that form a generally closed internal cavity within the implant body), entirely or partially open (i.e., sections of the implant body that are tubular in nature but are relatively open to the outside of the implant). The method may additionally or alternatively provide a means of operation for other types of electrical devices in conjunction with the implant. That is, beyond constructing an electrically stimulating enabled surgical implant, the method may be implemented to construct an implant enabled to function with specific electrical devices.

As this method may be implemented with any general implant construction method, dependent on implementation, method steps may be added, removed, or changed. For example, in some system implementations the implant may be constructed in advance for the addition of electrical components (i.e., pre-prepared) such that block S110 is not really implemented.

Block S110, which includes preparing an implant body, functions in procuring and prepping the desired implant for electrode enhancement. In some variations, the implant may be fully constructed prior to applying this method. This may be the case in implementations where a mundane surgical implant is to be enhanced.

Alternatively, block S110 may comprise constructing the implant body. Dependent on implementation, any desired method for acquisition or construction of the implant body may be incorporated (e.g., purchased, sculpted, molded, constructed using a CNC machine, 3D printed, etc.). In some variations, the implant body may be constructed in multiple pieces. These pieces may be combined directly or during other method steps. For example, a surgical nail may be constructed as two metal bodies that can be connected to each other. In another example, the spinal cage parts may be molded from plastic material (e.g., from PEEK).

The implant may be composed of any desired material. The implant is preferably constructed of the typical material for that implant. That is, the implant may be constructed of any biologically friendly, non-toxic material. The implant may be constructed of conductive materials (e.g., metals such as titanium or platinum) or non-conductive materials (e.g., plastics such as poly-ether ether ketone or silicone).

In variations for a spinal cage implant body, procuring an implant may comprise constructing a spinal cage as a single body, or multiple bodies (e.g., constructed using a CNC machine or molded). In one implementation of a spinal cage construction, as shown in FIGS. 31-38, block S110 includes constructing two side panels of the frame of the spinal cage, wherein each piece contains part of a “head” and “tail” piece of the implant body. These pieces may be immediately attached or attached during some other time of the method (e.g., after installation of electrodes).

Once the implant is procured, preparing the implant body S110 further includes prepping the implant body for electrical components. Prepping the implant body for electrical components may include establishing and building sites for the electrical components to be added, and electrical conduits for function of the electrical components. These include: establishing electrode sites on the frame of the implant such that electrodes can be fixed into, or onto, the implant body; establishing component sites such that other electrical components can be fixed into, or onto, the implant body; and establishing electrical conduits paths such that electrical conduits connecting to the electrical components can be fixed into, or onto, the implant body. Prepping the implant body for electrical components functions to enable electrical components, and the necessary electrical conduits, to be added to the implant body. In many variations, parts or all of block S110 may be performed simultaneously. Alternatively, prepping the implant body for electrical components may be performed after procuring the implant body.

Establishing electrode sites, component sites, and/or electrical conduits may comprise shaping regions of the frame of the surgical implant (e.g., through etching, melting, compressing, etc.) and/or constructing additions to regions of the implant body (e.g., by overmolding, welding, attaching, etc.) to enable addition of components. In variations where the implant body includes conductive regions (e.g., regions, or all, of the implant body are constructed of titanium), prepping the implant body for electrical components S120 may further include creating additional space such that an insulation layer may be placed prior to the addition of the electrical components.

Prepping the implant body for electrical components may include creating/designating electrode sites on or within the frame of the implant body. In some variations, creating/designating electrode sites may comprise initially creating/designating electrode sites while constructing the implant body. In one variation for implants constructed of PEEK (e.g., spinal cages), creating/designating the implant body electrode sites may occur by constructing them using a CNC machine during implant body construction. In another variation, creating/designating electrode sites may comprise shaping, or carving out the electrode sites. In another PEEK body variation, the electrode sites may be shaped and formed by melting or etching the PEEK after construction (e.g., by a laser). In metal implant variations, the etching or cutting the implant body may also be used to create/designate electrode sites.

In some variations, creating/designating electrode sites may comprise constructing raised platforms that protrude from the structure of the implant body, as shown in FIG. 38. The raised platforms may be shaped such that electrode pads (of the designated shape) may be positioned on the platforms. These raised platforms may be positioned anywhere on the implant, dependent on the desired implant implementation. Additionally or alternatively, creating electrode sites may comprise creating holes in the body of the implant, such that electrodes may be fixed into, or onto the implant body, exposed at these hole regions.

In one spinal cage example, creating electrode sites may comprise creating raised electrode platforms. These electrodes may be slightly elevated from the body of the implant (e.g., such that they stay exposed with additional filling/insulation layer(s) added to the implant), as shown in FIG. 38. The electrode platforms may be positioned both on the outside of the spinal cage and along the interior cavity of the spinal cage. In one implementation, four electrode sites are created on the exterior perimeter of the spinal cage and four electrode sites are created on the interior perimeter of the spinal cage.

In one surgical nail example, creating electrode sites may comprise etching the exterior of the surgical nail to demarcate electrode sites. In some implementations this may occur prior to adding a base insulations layer, such that the polymer mask is primarily covering the etched regions. In another surgical nail example, for a tubular surgical nail, holes may be cut in the surgical nail such that an electrode may fit through the surgical nail holes. In a third surgical nail example for a surgical nail that is open (e.g., FIG. 65), the electrode site may be etched on the interior wall of the surgical nail, thereby enabling an electrode placement in the interior of the nail.

Creating/designing electrode sites may further include creating pathways for electrical conduits. Pathways for electrical conduits comprise pathways along the structure of the implant that travel from electrode sites to the implant casing, where the implant circuitry will be place. Creating pathways for electrical conduits may further include creating pathways from other electrical components, on or within the implant, leading to the position where the casing would be attached to the implant. These pathways may include ramps (e.g., from elevated regions such as elevated electrode sites), indentations, and holes traveling through the implant. That is creating pathways for the electrical conduits may enable conduits that travel from the interior and/or exterior surfaces of the implant.

In one spinal cage example, creating a pathway for an electrical conduit may comprise creating an elevated platform for each electrode, creating a ramp from the elevated platform to the structure of the implant, and creating a narrow indentation along the structure, and creating a hole at the casing end of the structure (e.g., to enable the conduit to connect with the circuitry casing 120).

In one solid surgical nail example, creating a pathway for an electrical conduits may comprise etching the exterior of the surgical nail to demarcate electrical conduits, where these etchings travel from the electrode site along the surface of the nail to the head of the nail. In another surgical nail example, for a tubular surgical nail, electrical conduits may not be necessary as wires may be drawn through the hollow interior of the surgical nail. Additionally or alternatively, electrical conduits may be etched onto the interior surface of the tubular surgical nail.

Creating component sites may include creating regions for installation of other electrical components on, or within, the implant. Creating component sites may be implemented for the addition of any desired electrical components that may be used with the implant. In some variations, creating component sites may include creating antenna sites. For spinal cage variations, antenna sites may be created during construction of the spinal cage.

Block S120, which includes installing implant body electrical components, functions to fix the electrical components onto, or into, the structure of the implant body. Installing implant body electrical components comprises installing electrodes, other electrical components, and the actual “wiring” (or electrical conduits) necessary for their function. “Wiring” as used herein is used loosely to refer to any type of electrical connection (e.g., actual wire, electric pathway on a microchip, conductive metal pathway, etc.).

Dependent on the implant composition, conductive, or non-conductive, installing implant body electrical components S120 may occur prior to or after an implementation block S130. That is, if the implant, or a region of the implant, is composed of conductive material, over-coating the implant body S130 may be called prior block S120.

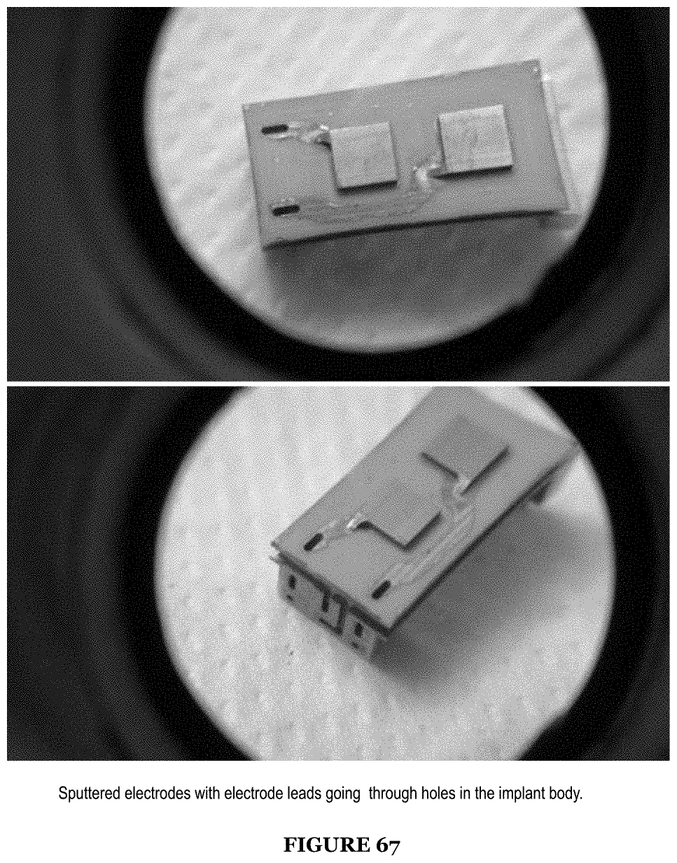

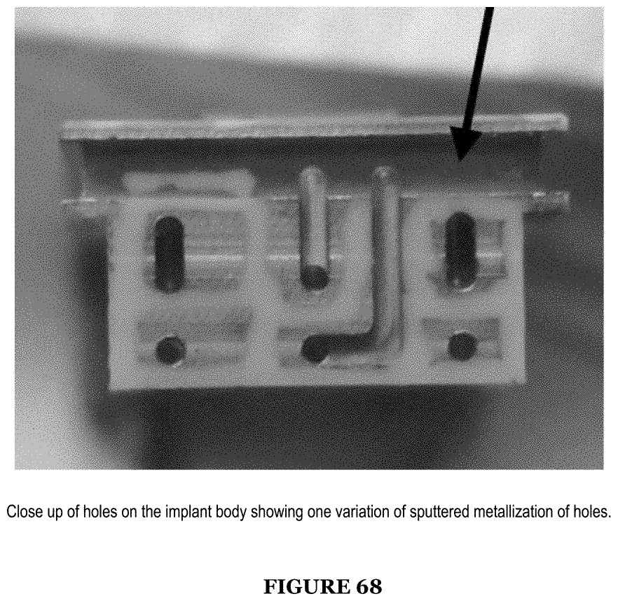

Installing implant body electrical components S130, may include installing electrodes. Installing electrodes may include positioning and/or locking the electrodes into the electrode sites. In some variations, the electrode may have 2 parts such that one part fits on a platform piece, and a second piece attaches underneath the platform to lock it in place. Additionally or alternatively, installing electrodes may include: gluing them into place, welding them into place (for example welding metal folds), etc. In one example, as shown in FIGS. 67 and 68, electrodes are incorporated by sputtering. In one implementation of this example, thin metal film is sputtered on the raised platform to form the electrode and a continuous path is sputtered along the path and through the implant hole to create the conductive lead to the implant circuitry. Alternatively, a metal sheet electrode (e.g., platinum) may be placed on the raised platform and a metal trace may be created (e.g., by sputtering) from the sheet electrode along the designated pathway.

Installing implant body electrical components S120, may include installing other electrical components. Examples of other electrical components may include: antennas, sensors, batteries, etc. In some spinal cage variations, antennas may be installed in the implant body. Laying the antenna may comprise, spiraling, winding, wrapping, and/or coiling the antenna around the perimeter of a part of the implant body. In some implementations, one antenna is wound around the perimeter of two parallel surfaces of the implant body.

Installing implant body electrical components S120 may include tracing electrical conduits. Tracing electrical conduits functions in placing conductive material along the prepared pathways for the electrical conduits. Any type of bio-friendly conductive material may be placed for tracing electrical conduits. Tracing electrical conduits may include: setting foil lines, sputtering wiring, installing physical wires, or any other desired conductive material. In some variations, tracing electrical conduits may occur simultaneously to creating electrical conduits. For example, chemical sputtering may be implemented to etch an electrical conduit while simultaneously depositing metal plate wiring. In some variations, tracing electrical conduits may deposit conductive material in different planes.

Block S130, which includes over-coating the implant, comprises adding a non-conductive coating to the surgical implant. Over-coating the implant S130 may serve multiple functions. These functions include: providing insulation to conductive components, giving an external shape to the surgical implant (e.g., adding grooves), connecting implant components (e.g., combining parts of a spinal cage, or connecting the circuitry casing 120 to implant body. Due to the multi-functionality of over-coating the implant S130, block S130 may be called before, after, or during and other method step as deemed necessary. As one particular important implementation, for implants that are composed of conductive material. Over-coating the implant S130 may lay an insulating layer over the conductive component prior to installing implant body electrical components S120.

Over-coating an implant S130 may be implemented to add an insulation layer. Dependent on implementation, this may include adding a base insulation layer (onto the implant base structure conductive components), adding any number of intermediary insulation layers (in between conductive components), and/or adding an outer insulation layer (above all components).

In variations where the implant body is mostly or entirely conductive, adding an insulation layer includes adding a base insulation layer. Adding a base insulation layer may include covering the entire implant body, or regions of the implant body, with an insulation material. In variations that include tubular, or cavity structures, adding a base insulation layer may include adding interior insulation layers, as deemed necessary for operation. That is, the base insulation layer may be added to both interior and exterior volumes of the implant as deemed necessary. Adding a base insulation layer may comprise printing a polymer mask onto the desired regions of the implant, wherein the polymer mask may be composed of any implant approved non-conductive material. Additionally or alternatively, the base insulation layer may comprise overmolding certain regions of the implant body. For example, for one surgical nail implementation, a polymer mask may be printed on the exterior surface of the surgical nail.

As electrode sites and electrical conduits may be situated on top of base insulation layer, adding a base insulation layer may be implemented in conjunction with block S110 to prep the implant. That is adding the base insulation layer may be simultaneously used to shape the implant surface and create electrode sites and pathways for electrical conduits.

In variations where conduits may pass through electrode sites, or multiple electrical components overlap, adding an insulation layer may include adding intermediary insulation layers. Adding intermediary insulation layer comprise adding insulation material between two conducting components. Any number of intermediary insulation layers may be added as deemed necessary. Adding an intermediary insulation layer may comprise printing a polymer mask onto the desired regions of the implant, wherein the polymer mask may be composed of any implant approved non-conductive material. Additionally or alternatively, the intermediary insulation layer may comprise overmolding certain regions of the implant body. For example, for one solid surgical nail implementation, a polymer mask may be printed on top of an electrode conduit and formed on the exterior surface of the surgical nail.

As electrode sites and electrical conduits may be situated on top of intermediary insulation layer, adding an intermediary insulation layer may be implemented in conjunction with block S110 to prep the implant. That is adding the intermediary insulation layer may be simultaneously used to shape the implant surface and create electrode sites and pathways for electrical conduits.

Adding an outer insulation layer may function to cover all conductive regions exposed on the implant that are not supposed to be exposed. That is, adding an outer insulation layer functions to cover all conducting regions. Preferably, adding an outer insulation layer does not add an insulation layer over electrode sites thereby enabling electrical stimulation. Adding an insulation layer may comprise printing a polymer mask onto the desired regions of the implant, wherein the polymer mask may be composed of any implant approved non-conductive material. For example, for one surgical nail implementation, a polymer mask may be printed on the exterior surface of the surgical nail. Additionally or alternatively, adding an outer insulation layer may comprise overmolding certain regions of the implant. As the outer insulation layer may comprise the “final” surface of the surgical implant. Adding an outer insulation layer may further include finishing the implant.

Over-coating the implant S130 may include finishing the implant, wherein finishing the implant may include adding additional structural and/or finishing features on the implant. For examples, this may include adding ridges or teeth to spinal cages to increase their surface friction. Adding additional pieces may be in conjunction with adding that an outer insulation layer. For example, a single overmolding process may be implemented to create a finalized shape of the implant, create additional external features, fill in conduit routing holes, lock a connector piece in place, etc. As finishing the implant may include modifying (e.g., overmolding) over the entire implant, over-coating the implant S130 may occur after any and all steps.

Block S140, which includes building an implant casing functions to create an external housing for electrical components. Building an implant casing S140 may function to build a metal casing (e.g., from titanium) and/or plastics (e.g., PEEK), or other materials (e.g., silicone). The type of casing may be dependent on implementation. For example, for structural durability, the casing may be composed of metals, for signal transfer the housing may be constructed of non-metals (e.g., PEEK), for flexibility the housing may be constructed of silicone. Building an implant casing S140 may include installing casing electrical components. In some variations the casing may be built around the casing electrical components (e.g., a PEEK casing may be overmolded over the casing electrical components or titanium casing may be constructed around the electrical components).

The electrical components may need to be specially positioned to fit into the casing due to limited size and other geometric limitations. In some variations, the casing electrical components may be fit onto a chip. Additionally, the PCB may be folded such that it fits within the casing.

Block S150, which includes connecting the casing to the implant body functions to enable the electrically enhanced implant. Connecting the casing to the implant body S150 includes physically and electrically connecting the casing to the implant, such that the implant electrodes become functional and that with the control circuitry 134 of the implant may provide the desired electric stimulation and/or any other type of operation.

Connecting the casing to the implant body S150 may include adding a connector piece. The connector piece may comprise a piece that attaches both to the implant (at a desired location) and to the casing. Dependent on variation, adding a connector piece may occur prior to or after overmolding the implant (or other means finishing the implant body). Dependent on implementation, the connector piece may include mechanisms or materials construction that enable it lock, fasten, meld (e.g., melt together), fuse (e.g., by welding), adhere, and/or attach in some other manner to both the implant body and to the casing. In spinal cage variations the connector piece may fit on top of the spinal cage and potentially help hold together multiple spinal cage pieces. In one implementation, the connector piece may have a rod-locking mechanism wherein a rod passes through the connector piece and each of the spinal cage halves to lock them together. Additionally, the connector piece may be at least partially constructed of titanium such that it can be welded to the titanium casing, thereby creating a hermetically sealed casing.

The connector piece may additionally have holes that the electrical wiring may pass through from the implant body to the casing. Additionally or alternatively, the connector piece may include glass or ceramic material for sealing wiring that passes through the connector piece.

As used herein, first, second, third, etc. are used to characterize and distinguish various elements, components, regions, layers and/or sections. These elements, components, regions, layers and/or sections should not be limited by these terms. Use of numerical terms may be used to distinguish one element, component, region, layer and/or section from another element, component, region, layer and/or section. Use of such numerical terms does not imply a sequence or order unless clearly indicated by the context. Such numerical references may be used interchangeable without departing from the teaching of the embodiments and variations herein.

As a person skilled in the art will recognize from the previous detailed description and from the figures and claims, modifications and changes can be made to the embodiments of the invention without departing from the scope of this invention as defined in the following claims.

We claim:

1. A system for a bio-implantable stimulation device comprising:
a circuitry casing comprising control circuitry and a casing connector;
an inner frame comprising a set of electrode sites;
a set of conductive paths on the inner frame, where each conductive path has a first portion on a surface of the electrode site and a second portion formed as an electrical conduit connecting the electrode site to a connector of the casing connector; and
an over-coating that is formed around the inner frame with the electrode sites exposed on a surface of the over-coating.

2. The system of claim 1, wherein the set of electrode sites are raised platform structures on the inner frame.

3. The system of claim 2, wherein the raised platform structures have a ramp from a top surface of the raised platform structure to a recessed surface of the inner frame; and wherein the second portion of the conductive path runs from the recessed surface up the ramp to the top surface of the raised platform structure.

4. The system of claim 2, further comprising a body connector attached to one end of the inner frame; and wherein the second portion of each conductive path is formed as an electrical conduit on the inner frame from the electrode site to a conductive contact point of the body connector; wherein the casing connector mechanically and conductively couples to the body connector.

5. The system of claim 4, wherein the circuitry casing is welded to the body connector.

6. The system of claim 1, wherein the circuitry casing comprises a metal body, and wherein the control circuitry is contained within the outer metal body.

7. The system of claim 2, wherein the over-coating is molded polyetheretherketone.

8. The system of claim 2, wherein the inner frame includes at least one antenna coil inset; and further comprising a wire coiled around the antenna coil inset, wherein two ends of the wire are conductively connected to the casing connector.

9. The system of claim 2, wherein the control circuitry is encased within a metal outer body of the circuitry casing, and wherein the control circuitry is arranged as a folded circuit system.

10. The system of claim 9, wherein the control circuitry is an at least partially flexible printed circuit board.

11. The system of claim 1, wherein the inner frame comprises a defined channel tunnel from a surface of a first electrode site of the set of electrode sites to a connection point to the casing connector, wherein a first conductive path of the set of conductive paths is conductively connected from the first electrode site to the connection point through the defined channel tunnel.

12. The system of claim 11, further comprising a body connector attached to one end of the inner frame; and wherein the second portion of each conductive path is formed as an electrical conduit on the inner frame from the electrode site to a conductive contact point of the body connector; wherein the casing connector mechanically and conductively couples to the body connector; and wherein the defined channel tunnel extends from one end of the second portion of the first conductive path to a first conductive contact point of the body connector.

13. The system of claim 11, further comprising a body connector attached to one end of the inner frame; and wherein the second portion of each conductive path is formed as an electrical conduit on the inner frame from the electrode site to a conductive contact point of the body connector; wherein the casing connector mechanically and conductively couples to the body connector; and wherein the defined channel tunnel is defined from a first side of the inner frame where the first electrode site is positioned to a second side of the inner frame where the second portion of the first conductive path is positioned, wherein a conductive connection is established between the first electrode site to the second portion of the first conductive path through the defined channel tunnel.

14. The system of claim 1, wherein the inner frame comprises a recessed channel defining a path between a first electrode site of the set of electrode sites to a connection point, wherein for a first conductive path of the set of conductive paths, the second portion of the first conductive path is formed by conductive material deposited into the recessed channel.

15. The system of claim 1, wherein the set of conductive paths are a conductive layer sputtered onto the inner frame.

16. The system of claim 1, wherein the set of conductive paths are patterned conductive foil adhered to surfaces of the inner frame.

17. The system of claim 1, wherein the inner frame is made of conductive material; and further comprising an insulating layer between the inner frame and the set of conductive paths.

18. The system of claim 1, wherein the inner frame is comprised of at least two side panels that combine to form the inner frame.

19. A system for a bio-implantable stimulation device comprising:
a circuitry casing with a casing connector;
an inner frame comprising a set of electrode sites, where the set of electrode sites are raised platform structures;
a body connector attached to one end of the inner frame and that electrically couples to the casing connector;
a set of conductive paths on the inner frame, where each conductive path has a first portion on a surface of the electrode site and an electrical conduit portion connecting the electrode site to a connector of the body connector and thereby connected to the control circuitry through the casing connector; and
over-coating that is formed around the inner frame with the set of electrode sites exposed on a surface of the over-coating.
